(19) United States
(12) Patent Application Publication
(10) Pub. No.: US 2012/0129773 A1
(43) Pub. Date: May 24, 2012

(54) METHODS OF TREATING AUTISM AND AUTISM SPECTRUM DISORDERS

(76) Inventors: Mark R. GEIER, Silver Spring, MD (US); David A. GEIER, Silver Spring, MD (US)

(21) Appl. No.: 13/338,008

(22) Filed: Dec. 27, 2011

Related U.S. Application Data
(63) Continuation of application No. 11/589,490, filed on Oct. 30, 2006.
Application No. 11/589,490 is a continuation-in-part of application No. 11/225,623, filed on Sep. 13, 2005, now abandoned.
Application No. 11/225,623 is a continuation-in-part of application No. 10/941,887, filed on Sep. 16, 2004, now abandoned.

(51) Int. Cl. A61K 38/09; A61P 15/00; A61P 5/30; A61P 25/00; A61P 5/28

(52) U.S. Cl. 514/9.7

(57) ABSTRACT

The present invention relates to methods of treating a subject diagnosed with autism or an autism spectrum disorder, lowering the level of mercury in a subject determined to contain a high level of mercury, methods of lowering the level of mercury in a child diagnosed with autism, lowering the level of at least one androgen in a subject diagnosed with autism, lowering the level of mercury and the level of at least one androgen in a subject diagnosed with autism and methods of assessing the risk of whether a child is susceptible of developing autism.

### RELATED APPLICATION INFORMATION

This application is a continuation-in-part of U.S. application Ser. No. 11/225,623 filed on Sep. 13, 2005, which is a continuation-in-part of U.S. application Ser. No. 10/941,887 filed on Sep. 16, 2004, the contents of which are herein incorporated by reference.

### FIELD OF THE INVENTION

The present invention relates to methods of treating a subject diagnosed with autism or an autism spectrum disorder, lowering the level of mercury in a subject determined to contain a high level of mercury, methods of lowering the level of mercury in a child diagnosed with autism, lowering the level of at least one androgen in a subject diagnosed with autism, lowering the level of mercury and the level of at least one androgen in a subject diagnosed with autism, lowering the level of mercury and the level of at lease one androgen and raising the level of estrogen in a subject diagnosed with autism, and methods of assessing the risk of whether a child is susceptible of developing autism.

### BACKGROUND OF THE INVENTION

Autism is a neurodevelopmental disorder characterized by impairments in social relatedness and communication, repetitive behaviors, abnormal movements, and sensory dysfunction. According to the most recent estimates published by the Centers for Disease Control and Prevention (CDC), it has been reported that approximately 1 in 150 children in the United States suffers from an autistic disorder, and far more males than females suffer from autistic disorders (See, Bertrand J, Mars A, Boyle C, Bove F, Yeargin-Allsopp M, Decoufle P., “Prevalence of autism in a United States population: the Brick Township, N.J., investigation,” Pediatrics 2001; 108:1155-61, Yeargin-Allsopp M, Rice C, Karapurkar T, Doernberg N, Boyle C, Murphy C., “Prevalence of autism in a US metropolitan area”, JAMA 2003; 289:49-55, Gerlai R, Gerlai J., “Autism: a target for pharmacotherapies?” Drug Discov Today 2004; 9:366-74, Gerlai R, Gerlai J., “Autism: a large unmet medical need and a complex research problem,” Physiol Behav, 2003; 79:461-70, California Department of Developmental Services. “Autistic Spectrum Disorders—Changes in the California Caseload—An Update: 1999 through 2002,” Sacramento, Calif.: State of California, 2003, Blaxill M F, Baskin D S, Spitzer W O., “Commentary: Blaxill, Baskin, and Spitzer on Croen et al. (2002), the changing prevalence of autism in California. J Autism Dev Disord 2003; 33:223-6, Blaxill M F. “What's going on? The question of time trends in autism,” Public Health Rep 2004; 119:536-51 and Newschaffer C J, Falb M D, Gurney J G., “National autism prevalence trends from United States special education data,” Pediatrics 2005; 115:e277-82). Furthermore, in recent research observing children's communicative, social, affective, repetitive behaviors, and toy play coded from videotapes of the toddlers' first and second birthday parties there are significant numbers of children with regressive autistic disorders that manifest between the ages of 12 and 24 months of age, a temporal period directly post-administration of mercury from thimerosal-containing childhood vaccines in the United States (See, Werner E, Dawson G., “Validation of the phenomenon of autistic regression using home videotapes,” Arch Gen Psychiatry 2005; 62:889-95).

Mercury toxicity has been reported throughout history. For example, mercury has been found in Egyptian tombs, indicating it was used as early as 1500 BC. In the late 18th century, antisyphilitic agents contained mercury. During the 1800's, the phrase “mad as a hatter” was coined because of the chronic mercury exposure that the felters faced because mercury was used in hat making. Today, humans are exposed to mercury from a variety of different sources, including dental amalgams, certain industries such as battery, thermometer and barometer manufacturing, ingestion of certain foods such as fish and shellfish, environmental pollution resulting from the use of fossil foods, prescription medicines, and from vaccinations and other biologicals, such as Rhₒ immune globulin, containing thimerosal, a mercury-containing preservative.

Mercury can be found in a variety of different forms. Elemental mercury can be found as a liquid or vapor. Organic mercury can be found in three different forms, aryl and short and long chain alkyl compounds. Examples of organic mercury include, but are not limited to, ethylmercury and methylmercury. Inorganic mercury is found mostly in the form of a mercuric salt, such as mercuric chloride. It is known in the art that mercuric chloride binds and forms a complex with testosterone in vitro and possibly in subjects (See, Cooper et al., “The Crystal Structure and Absolute Configuration of the 2:1 Complex between Tesosterone and Mercuric Chloride,” Acta Crystallogr B., 1968, 15:24(7):935-41).

Mercury toxicity or poisoning can result from vapor inhalation, ingestion, injection, or absorption through the skin. Exposure to any form of mercury on a repeated basis, or even from a single, very high exposure can lead to mercury toxicity or mercury poisoning. There are three main symptoms or mercury toxicity or mercury poisoning:
1. Gum problems. The gums become soft and spongy, the teeth get loose, sores may develop, and there may be increased saliva.
2. Mood and mental changes. People suffering from mercury toxicity or mercury poisoning often have wide swings of mood, becoming irritable, frightened, depressed or excited very quickly for no apparent reason. Such people may become extremely upset at any criticism, lose all self-confidence, and become apathetic. Hallucinations, memory loss and inability to concentrate can occur.
3. Nervous system. The earliest and most frequent symptom is a fine tremor (shaking) of the hand. A tremor may also occur in the tongue and eyelids. Eventually this can progress to trouble balancing and walking.

In addition, there are a number of other symptoms that may be caused by exposure to high levels of mercury and mercury-containing compounds. For example, skin allergies may develop. If this happens, repeated exposure causes rash and itching. Exposure to mercury vapor can cause the lens of the eye to discolor. In addition, some inorganic mercury compounds can cause burns or severe irritation of the skin and eyes on contact. Moreover, some organic mercury compounds (such as methylmercury and ethylmercury) are known to cause birth defects in children born of exposed mothers.

A number of diseases are believed to have a mercury toxicity component. These include autism, Alzheimer's disease (See, Pendergrass J C, Haley B E, Vimy M J, Winfield S A, Lorscheider F L, “Mercury vapor inhalation inhibits binding of GTP to tubulin in rat brain: similarity to a molecular lesion in Alzheimer diseased brain,” Neurotoxicology 1997; 18:315-24 and Pendergrass J C, Haley B E., “Inhibition of brain tubulin-guanosine 5′-triphosphate interactions by mercury: similarity to observations in Alzheimer's diseased brain,” Met Ions Biol Syst 1997; 34:461-78), diabetes (See, Waly M, Olteanu H, Banerjee R, Choi S W, Mason J B, Parker B S, Sukumar S, Shim S, Sharma A, Benzecry J M, Power-Chamitsky V A, Deth RC., “Activation of methionine synthase by insulin-like growth factor-1 and dopamine: a target for neurodevelopmental toxins and thimerosal,” Mol Psychiatry 2004; 9:358-70), heart disease (See, Guallar E, Sanz-Gallardo M I, van't Veer P, Bode P, Aro A, Gomez-Aracena J, Kark J D, Riemersma R A, Martin-Moreno J M, Kok F J; “Heavy Metals and Myocardial Infarction Study Group. Mercury, fish oils, and the risk of myocardial infarction,” N Engl J Med 2002; 347:1747-54), obesity (See, Kishida K, Kuriyama H, Funahashi T, Shimomura I, Kihara S, Ouchi N, Nishida M, Nishizawa H, Matsuda M, Takahashi M, Hotta K, Nakamura T, Yamashita S, Tochino Y, Matsuzawa Y., “Aquaporin adipose, a putative glycerol channel in adipocytes,” J Biol. Chem. 2000 Jul. 7; 275(27):20896-902), amyotrophic lateral sclerosis (ALS) (See, Sillevis Smitt P A, de Jong J M., “Animal models of amyotrophic lateral sclerosis and the spinal muscular atrophies,” J Neurol Sci 1989; 91:231-58 and Barber T E., “Inorganic mercury intoxication reminiscent of amyotrophic lateral sclerosis,” J Occup Med 1978; 20:667-9), asthma (See, Kazantzis G., “The role of hypersensitivity and the immune response in influencing susceptibility to metal toxicity,” Environ Health Perspect 1978; 25:111-8) and certain immune disorders (See, Nakagawa R., “Concentration of mercury in hair of diseased people in Japan,” Chemosphere 1995; 30:135-40).

Recent studies have reported that exposure to mercury can cause immune, sensory, neurological, motor, and behavioral dysfunctions similar to traits defining or associated with autism, and the similarities extend to neuroanatomy, neurotransmitters, and biochemistry (See, Bernard S, Enayati A, Redwood L, Roger H, Binstock T., “Autism: a novel form of mercury poisoning,” Med Hypotheses, 2001; 56:462-71, Bernard S, Enayati A, Roger H, Binstock T, Redwood L. “The role of mercury in the pathogenesis of autism,” Mol Psychiatry 2002; 7 Suppl 2:S42-3 and Blaxill M F, Redwood L, Bernard S., “Thimerosal and autism? A plausible hypothesis that should not be dismissed,” Med Hypotheses 2004; 62:788-94.).

Thimerosal, a preservative added to many vaccines, has become a major source of mercury among children in the United States who, within their first two years, may have received a quantity of mercury that exceeded Federal Safety Guidelines (See, Redwood L, Bernard S, Brown D., “Predicted mercury concentrations in hair from infant immunizations: cause for concern,” Neurotoxicology 2001; 22:691-7 and Ball L K, Ball R, Pratt RD, “An assessment of thimerosal use in childhood vaccines,” Pediatrics 2001; 107:1147-54). According to the CDC recommended immunization schedule in the United States during the 1990's, infants may have been exposed to 12.5 μg of mercury at birth, 62.5 μg of mercury at 2 months, 50 μg of mercury at 4 months, 62.5 μg of mercury at 6 months, and 50 μg of mercury at 18 months, for a total of 237.5 μg of mercury during the first 18 months of life, if all thimerosal-containing vaccines were administered (See, Redwood L, Bernard S, Brown D, “Predicted mercury concentrations in hair from infant immunizations: cause for concern,” Neurotoxicology 2001; 22:691-7 and Ball L K, Ball R, Pratt RD., “An assessment of thimerosal use in childhood vaccines,” Pediatrics 2001; 107:1147-54.).

Redwood et al. (See, Redwood L, Bernard S, Brown D, “Predicted mercury concentrations in hair from infant immunizations: cause for concern,” Neurotoxicology 2001; 22:691-7) have estimated hair mercury concentrations expected to result from the recommended CDC childhood immunization schedule during the 1990s utilizing a one compartment pharmacokinetic model. The authors determined that modeled hair mercury concentrations in infants exposed to vaccinal thimerosal were in excess of the Environmental Protection Agency (EPA)'s safety guidelines of 1 part-per-million (ppm) for up to the first 365 days, with several peak concentrations within this period. The inventors have evaluated doses of mercury from thimerosal-containing childhood vaccines administered in accordance with the recommended CDC childhood immunization schedule during the 1990s in comparison the EPA and the Food and Drug Administration (FDA) safety guidelines for the oral ingestion of methylmercury, a similar compound to ethylmercury. Geier et al., the inventors of the present invention, reported that children received instantaneous doses of mercury from thimerosal-containing childhood vaccines that were many-fold in excess of the Federal Safety Guidelines (See Geier M R, Geier D A., “Thimerosal in childhood vaccines, neurodevelopment disorders, and heart disease in the United States,” J Am Phys Surg 2003; 8:6-11 and Geier D A, Geier M R., “An assessment of the impact of thimerosal on neurodevelopmental disorders,” Pediatr Rehabil 2003; 6:97-102.). In evaluating the dose of mercury children received from thimerosal-containing vaccines in the US, when factoring in significant environmental exposure (i.e., mercury in breast milk), it has been estimated the mercury in thimerosal-containing vaccines represented almost 50% of the total mercury dose infants received (See, Bighami M, Copes R. “Thiomersal in vaccines: balancing the risk of adverse effects with the risk of vaccine-preventable disease,” Drug Saf, 2005; 28:89-101.). As a result, it has been determined that some infants receiving 187.5 μg of mercury from thimerosal-containing vaccines during the first sixth months of life from the routine childhood vaccination schedule, in combination with environmental exposure from mercury in breast milk (164 μg of mercury), were exposed to cumulative doses of mercury during the first sixth months of life in excess of the methylmercury safety guidelines established by the EPA, Health Canada, the World Health Organization (WHO), the Agency for Toxic Substances Disease Registry (ATSDR), and the FDA. It was also determined that these same infants (with no additional exposure to mercury from any source) were in excess of the methylmercury guidelines established by the EPA, Health Canada, WHO, and the ATSDR for the entire first year of life.

In evaluating the distribution of mercury within the body following thimerosal-containing vaccine administration to infants, Burbacher et al. have evaluated infant monkeys following injection of doses of mercury comparable to the US dosing schedule (weight- and age-adjusted) (See, Burbacher T M, Shen D D, Liberato N, Grant K S, Cernichiari E, Clarkson T W., “Comparison of blood and brain mercury levels in infant monkeys exposed to methylmercury or vaccines containing Thimerosal,” Environ Health Perspect 2005; 113:1015-21). These researchers confirmed that thimerosal crosses the blood-brain barrier and results in appreciable mercury content in tissues including the brain (the maximum concentration observed in the brain was approximately 50-parts-per-billion). They determined that the overall half-life of mercury in the brain of the infant monkeys examined was approximately 24 days. In addition, it was determined that the percentage of inorganic mercury in the brains of the thimerosal-treated infant monkeys averaged 16 parts-per-billion following the dosing schedule, and the half-life of this inorganic mercury was found to be very long in the monkey brains (>120 days).

Furthermore, Hornig et al. administered thimerosal to mice, mimicking the United States' routine childhood immunization schedule of the 1990s (weight- and age-adjusted), and observed autistic symptoms in a susceptible mouse strain that included growth delay, reduced locomotion, exaggerated response to novelty, increased brain size, decreased numbers of Purkinje cells, significant abnormalities in brain architecture, affecting areas sub-serving emotion and cognition, and densely packed hyperchromic hippocampal neurons with altered glutamate receptors and transporters (See, Hornig M, Chian D, Lipkin W I., “Neurotoxic effects of postnatal Thimerosal are mouse strain dependent,” Mol Psychiatry 2004; 9:833-45). In addition, Digar et al. showed exposure to thimerosal from injection of a single 50 μg of mercury dose at specific prenatal developmental stages in an animal model resulted in significant fetal lethality and teratogenecity compared to controls (See, Digar A, Sensharma G C, Samal S N., “Lethality and teratogenecity of organic mercury (thimerosal) on the chick embryo,” J Anat Soc India 1987; 36:153-9).

In a series of molecular studies with neurons it has now been shown that nanomolar (nM) to micromolar (μM) concentrations of thimerosal are capable of inducing neuronal death, neurodegeneration, membrane damage, and DNA damage within hours of exposure (Baskin D S, Ngo H, Didenko V V., “Thimerosal induces DNA breaks, caspase-3 activation, membrane damage, and cell death in cultured human neurons and fibroblasts,” Toxicol Sci, 2003; 74:361-8, Parry J M., “An evaluation of the use of in vitro tubulin polymerisation, fungal and wheat assays to detect the activity of potential chemical aneugens,” Mutation Res 1993; 287:23-8, Wallin M, Hartely-Asp B., “Effects of potential aneuploidy inducing agents on microtubule assembly in vitro,”. Mutation Res 1993; 287:17-22, Brunner M, Albertini S, Wurgler F E., “Effects of 10 known or suspected spindle poisons in the in vitro porcine brain tubulin assembly assay,” Mutagenesis 1991; 6:65-70, James S J, Slikker W 3rd, Melnyk S, New E, Pogribna M, Jernigan S., “Thimerosal neurotoxicity is associated with glutathione depletion: protection with glutathione precursors,” Neurotoxicology 2005; 26:1-8 and Humphrey M L, Cole M P, Pendergrass J C, Kiningham K K., “Mitochondrial mediated thimerosal-induced apoptosis in a human neuroblastoma cell line (SK-N-SH),” Neurotoxicology 2005; 26:407-16). Additionally, it has also been shown that nM to μM concentrations of thimerosal are capable of disrupting critical signaling pathways/biochemical events necessary for neurons to undergo normal neuronal development (See, Parran D K, Barker A, Ehrich M., “Effects of Thimerosal on NGF signal transduction and cell death in neuroblastoma cells,” Toxicol Sci 2005; 86:132-40, Waly, et al., “Activation of methionine synthase by insulin-like growth factor-1 and dopamine: a target for neurodevelopmental toxins and thimerosal,” Mol Psychiatry 2004; 9:358-70 and Mutkus L, Aschner J L, Syversen T, Shanker G, Sonnewald U, Aschner M., “In vitro uptake of glutamate in GLAST- and GLT-1-transfected mutant CHO-K1 Cells is inhibited by the ethylmercury-containing preservative thimerosal,” Biol Trace Elem Res 2005; 105:71-86).

Epidemiological studies conducted in the United States have examined the relationship between thimerosal-containing childhood vaccines and autistic and other neurodevelopmental disorders. It has been shown that children receiving thimerosal-containing childhood vaccines were 2- to 8-fold statistically significantly more likely to develop autistic and other neurodevelopmental disorders, depending upon the symptoms and outcomes examined, in comparison to children receiving thimerosal-free childhood vaccines (See, Geier M R, Geier D A., “Thimerosal in childhood vaccines, neurodevelopment disorders, and heart disease in the United States,” J Am Phys Surg 2003; 8:6-11, Geier D A, Geier M R., “An assessment of the impact of thimerosal on neurodevelopmental disorders,” Pediatr Rehabil 2003; 6:97-102, Geier M R, Geier D A., “Neurodevelopmental disorders after thimerosal-containing vaccines: a brief communication,” Exp Biol Med 2003; 228:660-4 and Geier D A, Geier M R, “A comparative evaluation of the effects of MMR immunization and mercury doses from thimerosal-containing childhood vaccines on the population prevalence of autism,” Med Sci Monit 2004; 10(3):PI33-9, Geier D A, Geier M R., “Neurodevelopmental disorders following thimerosal-containing childhood immunizations: a follow-up analysis,” Int J Toxicol 2004; 23:369-76, Geier D A, Geier M R., “A two-phased population epidemiological study of the safety of thimerosal-containing vaccines: a follow-up analysis,” Med Sci Monit 2005; 11(4):CR160-70.).

Several recent studies have clinically evaluated the body-burden of heavy metals present in children with autistics disorders in comparison to normal children. Bradstreet et al. (See, Bradstreet J, Geier D A, Kartzinel J J, Adams J B, Geier M R, “A case-control study of mercury burden in children with autistic spectrum disorders,” J Am Phys Surg 2003; 8:76-9) have evaluated urinary heavy metals following three days of oral chelation with meso-2,3-dimercaptosuccinic acid (DMSA) in children with autistic disorders in comparison to a control population. It was determined that autistic children had statistically significantly approximately 6-fold higher urinary mercury concentrations than matched normal controls, whereas other heavy metals were present in similar urinary concentrations in both groups following three days of oral chelation with DMSA. In addition, in this study, urinary mercury concentrations were compared following three days of oral chelation with DMSA in matched vaccinated and unvaccinated normal children. It was observed that there were similar concentrations of urinary mercury in both groups following DMSA treatment. Holmes et al. (See, Holmes A S, Blaxill M F, Haley B E., “Reduced levels of mercury in first baby haircuts of autistic children,” Int J Toxic 2003; 22:277-85) have evaluated first baby haircuts from autistic children in comparison to controls. It was observed that the mercury levels in the first baby haircuts of children were inversely related to the severity of the autistic disorders of the children (i.e. the more severely affected the children are, the less mercury levels were present in their first baby haircuts). It has been hypothesized that these results are consistent with autistic children having biochemical differences than normal children, possibly as a result of genetic polymorphisms, resulting in children with autistic disorders having an increased body-burden of mercury in comparison to normal children.

James et al. (See, James S J, Culter P, Melnyk S, et al., “Metabolic biomarkers of increased oxidative stress and impaired methylation capacity in children with autism” Am Clin Nutr 2004; 80:1611-7) have evaluated the methionine cycle and transsulfuration metabolites in autistic children in comparison age- and sex-matched control children. It was determined that there were significant decreases in the plasma concentration of cysteine (19% reduction) and total glutathione (46% reduction), both of which are crucial for mercury excretion, in autistic children in comparison to control children. Additionally, consistent with the DMSA treatment and first baby haircut study results, it was determined that autistic children had significantly increased oxidative stress (3-fold decrease in total glutathione/oxidized glutathione redox ratio) in comparison to control children.

Boris et al. (See, Boris M, et al., “Association of 5,10-Methylenetetrathydrofolate reductase (MTHFR) gene polymorphisms with autistic spectrum disorders,” J Am Phys Surg 2004; 9:106-8 recently conducted genomic studies of children with autistic disorders in comparison to normal control populations. The authors examined genes in pathways that are responsible for the synthesis of key biochemical molecules that are of functional relevance in the excretion and/or oxidative stress protection of mercury from the body. Notably, only 2% of children with autistic disorders examined by the authors did present with at least one single nucleotide polymorphism (SNP) in the MTHFR gene. Additionally, the authors demonstrated that there was approximately a 2-fold statistically significant increase in both the homozygous (677 TT) and heterozygous (677 CT and 1298 AC) SNPs in the MTHFR gene in autistics in comparison to controls. This is of particular relevance because MTHFR is one of the key genes in the biochemical pathway involved with the synthesis of glutathione, a key molecule in the body's natural defenses against mercury, and those with homozygous (677 TT) or heterozygous (677 CT and 1298 AC) SNPs in the MTHFR gene have been found to have an enzyme that functions approximately 50-60% less than those with the wild-type MTHFR gene.

The understanding of the cause of the epidemic has allowed for the design of treatment modalities that address the mercury toxic component of these disorders. These therapies include methods to remove the mercury by such techniques as the use of chelating agents and by corrections in various biochemical pathways that lead to sulphydral-containing compounds that the body uses to rid itself of the mercury (See, Johnson S., “Micronutrient accumulation and depletion in schizophrenia, epilepsy, autism and Parkinson's disease?” Med Hypotheses 2001; 56:641-5.).

Haley (See, Haley BE. Mercury toxicity: genetic susceptibility and synergistic effects. Med Ver 2005; 2:535-42) has shown in tissue culture that mercury induced neuronal damage is exacerbated by concurrent exposure with testosterone, whereas mercury induced neuronal damage was ameliorated by concurrent exposure with estrogen. Clarkson et al. (See, Clarkson T W, Nordberg G F, Sager P R. Reproductive and developmental toxicity of metals. Scand J Work Environ Health. 1985; 11:145-54) have developed a mouse model to evaluate the neurotoxic effects of alkyl mercury exposure on different sexes. The authors reported that two-day-old mice were administered alkyl mercury at 4 mg of mercury/kg/bodyweight (low dose), 8 mg of mercury/kg/bodyweight (high dose), or nor mercury. Animals were sacrificed 24 hours later, and matched sections of brain were prepared. The total number of mitotic figures in the external granule layer of the cerebellar cortex were recorded and classified as early (prophase and metaphase) or late (anaphase and telophase). Mercury concentrations in the brain for both males and females were 2.7 micrograms of mercury/gram at the high dose exposure and 1.8 micrograms of mercury at the low dose exposure. The authors determined that at the high dose, male and female mice had similarly reduced percentages of late mitotic figures compared with controls. At the lower dose, female mice were significantly much less affected in their percentages of late mitotic figures compared with male mice. The authors concluded males are considerably more sensitive to the neurotoxic effects of mercury, and that in some human fetal/infant population exposures to low dose alkyl mercury, it has been observed that males were more sensitive than females to psychomotor retardation (See, Clarkson T W, Nordberg G F, Sager P R., “Reproductive and developmental toxicity of metals,” Scand J Work Environ Health. 1985; 11:145-54 and Grandjean P, Weihe P, White R F, Debes F., “Cognitive performance of children prenatally exposed to “safe” levels of methylmercury,” Environ Res 1998; 77:165-72) Muraoka and Itoh (Muraoka Y, Itoh F., “Sex difference of mercuric chloride-induced renal tubular necrosis in rats—from the aspect of sex differences in renal mercury concentration and sulfhydryl levels—,” J Toxicol Sci 1980; 5:203-14) have investigated sex differences in the effects of mercury exposure on other organ systems. The authors reported that when doses of 0.3 to 2 mg/kg of mercuric chloride were intravenously administered to rats of the JCL-SD strain, acute renal tubular necrosis was produced in the straight portion of the proximal tubules with a pronounced sex difference, the male being more susceptible. Necrosis was inhibited by castration of male rats and promoted by testosterone pretreatment.

Additionally, estrogens have been shown to themselves raise glutathione levels and thus may be of help to the patients being treated. (See, Oliveira F R, Ferreira J R, dos Santos C M, Macedo L E, de Oliveira R B, Rodrigues J A, do Nascimento J L, Faro L R, Diniz D L. Estradiol reduced cumulative mercury and associated disturbances in the hypothalamus-pituitary axis of ovariectomized rats. Exotoxicol Environ Saf2006; 63:488-93. Olivieri G, Novakovic M, Savaskan E, Meier F, Baysang G, Brockhaus M, Muller-Spahn F. The effects of beta-estradiol on SHSY5Y neuroblastoma cells during heavy metal induced oxidative stress, neurotoxicity and beta-amyloid secretion. Neuroscience 2002; 113:849-55).

Researchers (See, Manning J T, Baron-Cohen S, Wheelwright S, Sanders G., “The 2nd to 4th digit ratio and autism,” Dev Med Child Neurol 2001; 43:160-4 and Lutchmaya S, Baron-Cohen S, Raggatt P, Knickmeyer R, Manning J T., “2nd to 4^{th} digit ratios, fetal testosterone and estradiol,” Early Hum Dev 2004; 77:23-8) have investigated prenatal testosterone levels in children with autistic spectrum disorders. The authors examined 72 children with autism, including 23 children with Aspergers syndrome (i.e. these children have less serve autistic affects), 34 siblings, 88 fathers, 88 mothers, and sex and age-matched controls. The authors demonstrated that the more severely affected the children were the higher the levels of prenatal testosterone.

Additionally, other researchers (See, Geier D A, Geier M R. A clinical and laboratory evaluation of methionine and androgen pathway markers in children with autistic disorders. Horm Res 2006; 66:182-8) have found significantly elevated postnatal androgen levels in autistic disorders, and have observed an apparent interaction between significant decreases among metabolites in the methionine cycle-transsulfuration pathways and significant increases among metabolites in the androgen pathway. Furthermore, it has specifically been shown that the conversion of dehydroepiandrosterone (DHEA) to dehydroepiandrosterone-sulfate (DHEA-S) by the enzyme hydroxysteroid sulfotransferase (HST) is dependent upon sulphation, and the enzyme is inhibited by inflammation and viruses (See, Kim M S, Shigenaga J, Moser A, Grunfeld C, Feingold K R, “Suppression of DHEA Sulfotransferase (Sult2A1) during the Acute Phase Response,” Am. J. Physiol. Endocrinol. Metab., 2004; 287:E731-8).

Currently, there is a need in the art for new methods of treating subjects that are diagnosed with autism or autism spectrum disorders. Additionally, there is also a need in the art for new methods of treating subjects diagnosed with high levels of mercury and who suffer from mercury poisoning. Furthermore, there is also a need in the art for methods of treating subjects diagnosed with diseases or disorders that have a mercury component. Also, there is also a need in the art for methods of treating subjects exhibiting a high level of one or more androgens and who suffer from autism or autism spectrum disorders. Moreover, there is also a need in the art for methods of treating subjects exhibiting a high level of mercury and a high level of one or more androgens and are diagnosed with autism or an autism spectrum disorder.

### SUMMARY OF THE PRESENT INVENTION

In one embodiment, the present invention relates to methods of lowering the level of mercury in a subject diagnosed or suffering from mercury toxicity. The method can have the following steps:

a) administering to said subject a pharmaceutically effective amount of at least one luteinizing hormone releasing hormone composition; and

b) repeating step a) as necessary to lower the level of mercury in said subject.

Alternatively, the method can have the following steps:

a) administering to said subject a pharmaceutically effective amount of at least one luteinizing hormone releasing hormone composition;

b) administering to said subject a pharmaceutically effective amount of at least one chelating agent; and

c) repeating step a) or step b) or step a) and step b) as necessary to lower the level of mercury in said subject.

The methods described above can also further optionally comprise the step of administering to the subject a pharmaceutically effective amount of at least one antiandrogenic hormone either prior to or after step a), step b) or step a) and step b) (in the first method described above) or after step a), step b), step c), step a) and step b), step a) and step c), step b) and step c) or step a), step b) and step c) (in the second method described above). Administration of the at least one antiandrogenic hormone can be repeated as necessary to lower the level of mercury in the subject.

The methods described above can also further optionally comprise the step of administering a pharmaceutically effective amount of at least one androgen compound either prior to or after step a), step b) or step a) and step b) (in the first method described above) or after step a), step b), step c), step a) and step b), step a) and step c), step b) and step c) or step a), step b) and step c) (in the second method described above).

Administration of the at least one androgen can be repeated as necessary to lower the level of mercury in the subject.

The method described above can also further optionally comprise the step of administering to the subject (if the subject is a female who is of pubertal age) a pharmaceutically effective amount of at least one estrogen compound either prior to or after step a), step b) or step a) and step b) (in the first method described above) or after step a), step b), step c), step a) and step b), step a) and step c), step b) and step c) or step a), step b) and step c) (in the second method described above). Administration of the at least one estrogen compound can be repeated as necessary to lower the level of mercury in the subject. Optionally, either along with the at least one estrogen compound or separately, at least one progesterone compound or at least one progestin compound can be administered to said subject. The administration of the at least one progesterone compound or at least one progestin compound is repeated as necessary to lower the level of mercury in the subject.

The at least one luteinizing hormone composition used in the above-described methods can be a luteinizing hormone releasing hormone (“LHRH”) analogue, a LHRH agonist, a LHRH antagonist or combinations thereof. The at least one chelating agent that can be administered pursuant to the second method described above can be administered orally, transdermally, intravenously, orally and transdermally, orally, transdermally and intravenously, orally and intravenously or transdermally and intravenously. The at least one antiandrogenic hormone, if used in the above-described methods, can be cyproterone acetate, finasteride, bicalutamide, novaldex, nilandron, flutamide, progesterone, spironolactone, fluconazole or combinations thereof.

The subject treated pursuant to the above-described methods can be a human male or female, adult or child. If the subject being treated is a child, said child can have an age between two (2) years old and seventeen (17) years old. In addition, the human male or human female subject may also be suffering from a disorder selected from the group consisting of: autism, autism spectrum disorders, attention deficit disorder, attention deficit hyperactivity disorder, mental retardation, Asperger's syndrome, childhood psychoses, stammering, stuttering, tics, repetitive movements, eating disorders, sleep disorders, enuresis, developmental language disorders, developmental speech disorders, developmental delay, Alzheimer's disease, diabetes, heart disease, obesity, amyotrophic lateral sclerosis, nephritic syndrome, renal failure, asthma, systemic lupus, autoimmune thyroiditis, rheumatoid arthritis, arthritis, vasculities, myelitis, glomerulonephritis, optic neuritis, infantile cerebral palsy, epilepsy, schizophrenia, migraine, toxic encephalopathy, cerebral degenerations, anterior horn cell disease, spinocerebellar disease, extrapyramidal disease or myopathy. Preferably, the subject is suffering from autism, more preferably, the subject is a male child who has autism. Even more preferably, the subject is a male child, who has autism and who has also been diagnosed with precocious puberty.

In another embodiment, the present invention relates to methods of lowering the level of mercury in a subject suffering from mercury toxicity, wherein said subject also suffers from autism. The method can involve the following steps:

a) administering to said subject a pharmaceutically effective amount of at least one luteinizing hormone releasing hormone composition; and

b) repeating step a) as necessary to lower the level of mercury in said subject.

Alternatively, the method can involve the following steps:

a) administering to said subject a pharmaceutically effective amount of at least one luteinizing hormone releasing hormone composition;

b) administering to said subject a pharmaceutically effective amount of at least one chelating agent; and

c) repeating step a) or step b) or step a) and step b) as necessary to lower the level of mercury in said subject.

The methods described above can also further optionally comprise the step of administering to the subject a pharmaceutically effective amount of at least one antiandrogenic hormone either prior to or after step a), step b) or step a) and step b) (in the first method described above) or after step a), step b), step c), step a) and step b), step a) and step c), step b) and step c) or step a), step b) and step c) (in the second method described above). Administration of the at least one antiandrogenic hormone can be repeated as necessary to lower the level of mercury in said subject.

The methods described above can also further optionally comprise the step of administering a pharmaceutically effective amount of at least one androgen compound either prior to or after step a), step b) or step a) and step b) (in the first method described above) or after step a), step b), step c), step a) and step b), step a) and step c), step b) and step c) or step a), step b) and step c) (in the second method described above). Administration of the at least one androgen can be repeated as necessary to lower the level of mercury in the subject.

The method described above can also further optionally comprise the step of administering to the subject (if the subject is a female who is of pubertal age) a pharmaceutically effective amount of at least one estrogen compound either prior to or after step a), step b) or step a) and step b) (in the first method described above) or after step a), step b), step c), step a) and step b), step a) and step c), step b) and step c) or step a), step b) and step c) (in the second method described above). Administration of the at least one estrogen compound can be repeated as necessary to lower the level of mercury in the subject. Optionally, either along with the at least one estrogen compound or separately, at least one progesterone compound or at least one progestin compound can be administered to said subject. The administration of the at least one progesterone compound or at least one progestin compound is repeated as necessary to lower the level of mercury in the subject.

The at least one luteinizing hormone composition used in the above-described methods can be a luteinizing hormone releasing hormone (“LHRH”) analogue, a LHRH agonist, a LHRH antagonist or combinations thereof. The at least one chelating agent that can be administered pursuant to the second method described above can be administered orally, transdermally, intravenously, orally and transdermally, orally, transdermally and intravenously, orally and intravenously or transdermally and intravenously. The at least one antiandrogenic hormone, if used in the above-described methods, can be cyproterone acetate, finasteride, bicalutamide, novaldex, nilandron, flutamide, progesterone, spironolactone, fluconazole or combinations thereof.

The subject treated pursuant to the above-described methods can be a human male or female, adult or child. If the subject is a child, the child treated pursuant to these methods can have an age between two (2) years old and seventeen (17) years old. Preferably, the subject is a male child, who has autism and who has also been diagnosed with precocious puberty.

In a fourth embodiment, the present invention relates to a method of assessing the risk of whether a subject (such as a human child or adult (male or female)) is susceptible of developing autism or autism spectrum disorders. The method involves the following steps:

a) determining the level of at least one androgen from a test sample obtained from a subject; and

b) assessing, based on a comparison of at least one androgen in said test sample with a reference level for said at least one androgen, whether said subject is at risk of developing autism.

In the above-described method, a subject is at risk of developing autism when said subject exhibits a level of at least one androgen that is at the reference level or greater than the reference level for at least one androgen for a subject of approximately the same age. In contrast, a subject is not at risk of developing autism when said subject has a level of at least one androgen that is lower than the reference level for said at least one androgen for a subject of approximately the same age.

In still yet another embodiment, the present invention relates to a method of treating a subject suffering from autism or an autism spectrum disorder, wherein said subject has an elevated level of at least one androgen (such as, but not limited to, an increase in serum testosterone or an elevated level of free serum testosterone) when compared to a reference level for said at least one androgen in a subject of approximately the same age. The method comprises the steps of:

a) administering to said subject a pharmaceutically effective amount of at least one luteinizing hormone releasing hormone composition; and

b) repeating step a) as necessary to lower the level of said at least one androgen in said subject and treat said autism or autism spectrum disorder.

Alternatively, the method can have the following steps:

a) administering to said subject a pharmaceutically effective amount of at least one luteinizing hormone releasing hormone composition;

b) administering to said subject a pharmaceutically effective amount of at least one chelating agent; and

c) repeating step a), step b) or step a) and step b) as necessary to lower the level of said at least one androgen in said subject and treat said subject.

The methods described above can also further optionally comprise the step of administering to the subject a pharmaceutically effective amount of at least one antiandrogenic hormone either prior to or after step a), step b) or step a) and step b) (in the first method described above) or after step a), step b), step c), step a) and step b), step a) and step c), step b) and step c) or step a), step b) and step c) (in the second method described above). Administration of the at least one antiandrogenic hormone can be repeated as necessary to treat the subject.

The methods described above can also further optionally comprise the step of administering a pharmaceutically effective amount of at least one androgen compound either prior to or after step a), step b) or step a) and step b) (in the first method described above) or after step a), step b), step c), step a) and step b), step a) and step c), step b) and step c) or step a), step b) and step c) (in the second method described above). Administration of the at least one androgen can be repeated as necessary to treat the subject.

The method described above can also further optionally comprise the step of administering to the subject (if the subject is a female who is of pubertal age) a pharmaceutically effective amount of at least one estrogen compound either prior to or after step a), step b) or step a) and step b) (in the first method described above) or after step a), step b), step c), step a) and step b), step a) and step c), step b) and step c) or step a), step b) and step c) (in the second method described above). Administration of the at least one estrogen compound can be repeated as necessary to treat the subject. Optionally, either along with the at least one estrogen compound or separately, at least one progesterone compound or at least one progestin compound can be administered to said subject. The administration of the at least one progesterone compound or at least one progestin compound is repeated as necessary to treat the subject.

The at least one luteinizing hormone composition used in the above-described methods can be a luteinizing hormone releasing hormone (“LHRH”) analogue, a LHRH agonist, a LHRH antagonist or combinations thereof. The at least one chelating agent that can be administered pursuant to the second method described above can be administered orally, transdermally, intravenously, orally and transdermally, orally, transdermally and intravenously, orally and intravenously or transdermally and intravenously. The at least one antiandrogenic hormone, if used in the above-described methods, can be cyproterone acetate, finasteride, bicalutamide, novaldex, nilandron, flutamide, progesterone, spironolactone, fluconazole or combinations thereof.

The subject treated pursuant to the above-described methods can be a human male or female, adult or child. If the subject being treated is a child, said child can have an age between two (2) years old and seventeen (17) years old. Preferably, the subject is a male child who has autism. More preferably, the subject is a male child, who has autism and who has also been diagnosed with precocious puberty.

### BRIEF DESCRIPTION OF THE DRAWINGS

FIG. 1 shows a description of the precursors to testosterone and estrogen in the steroidgenic pathway.

FIG. 2 shows the role of transsulfuration metabolites in the testosterone pathway.

FIG. 3 shows the breakdown pathway for testosterone.

FIG. 4 shows the interaction between androgen metabolites and methionine cycle-transsulfuration pathways.

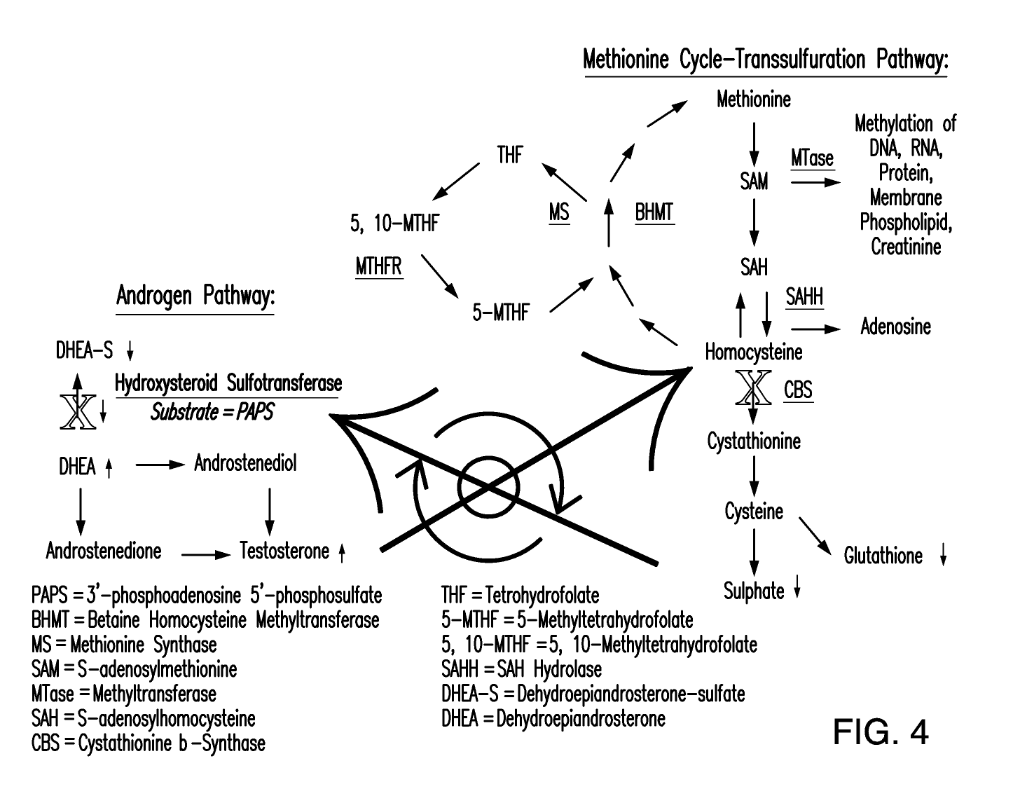

### DETAILED DESCRIPTION OF THE INVENTION

### Definitions

The terms “administer”, “administering”, “administered” or “administration” refer to any manner of providing a drug or pharmaceutically active agent (such as, at least one luteinizing hormone releasing hormone composition, at least one chelating agent, at, least one antiandrogenic hormone, etc.) to a subject or patient. Routes of administration can be accomplished through any means known by those skilled in the art. Such means include, but are not limited to, oral, buccal, intravenous, subcutaneous, intramuscular, by inhalation, transdermal and the like.

As used herein, the term “androgen” refers to any natural or synthetic compound that stimulates or controls the development and maintenance of masculine characteristics in a subject by binding to one or more androgen receptors. Examples of androgens include, but are not limited to, testosterone, DHEA, androstenedione, androstanediol, androsterone and dihydrotestosterone (DHT).

As used herein, the term “antiandrogenic hormone” refers to any pharmaceutically acceptable active agent that inhibits competitively the effect of androgens at their target site of action. Examples of antiandrogenic hormones that can be used in the present invention include, but are not limited to, cyproterone acetate, finasteride, bicalutamide, novaldex, nilandron, flutamide, progesterone, spironolactone, fluconazole or combinations thereof.

As used herein, the term “chelating agent” refers to any pharmaceutically active agent that is capable of binding or bonding to a mineral or metal present in a subject and then carrying that mineral or metal through the bloodstream to be excreted in the urine of said subject. Chelating agents can be administered to a subject orally, intravenously, subcutaneously, intramuscularly, transdermally, etc. Examples of chelating agents that can be used in the present invention include, but are not limited to, ethylenediaminetetraacetic acid (EDTA), DMSA, sodium dimercaptopropanesulfonate (DMPS), monoisoamyl DMSA (MiADMSA), etc.

By an “effective amount” or a “pharmaceutically effective amount” of a drug or pharmaceutically active agent, such as, at least one luteinizing hormone releasing hormone composition, at least one chelating agent, at least one antiandrogenic hormone, etc., is meant a nontoxic but sufficient amount of the drug or pharmaceutically active agent to provide the desired effect. The amount of drug or pharmaceutically active agent that is “effective” will vary from subject to subject, depending on the age and general condition of the individual, the particular drug or pharmaceutically active agent and the like. Thus, it is not always possible to specify an exact “effective amount.” However, an appropriate “effective amount” in any individual case may be determined by one of ordinary skill in the art using routine experimentation.

The term “gonadotropin” or “gonadotropins” refers to protein hormones secreted by gonadotrope cells of the pituitary gland of mammals. The two principal gonadotropins are luteinizing hormone (“LH”) and follicle stimulating hormone (“FSH”).

The term “luteinizing hormone releasing hormone” (also known as “gonadotropin-releasing hormone” or “GNRH”) or “LHRH” refers to hormone that is a decapeptide having the following structure:

(Pyr)-Glu-His-Trp-Ser-Tyr-Gly-Leu-Arg-Pro-Gly-NH₂

The term “luteinizing hormone releasing hormone composition” or “LHRH composition” refers to a LHRH (or GNRH) analogue, a LHRH (or GNRH) agonist, a LHRH (or GNRH) antagonist or any combination of a LHRH analogue, LHRH agonist or LHRH antagonist that is capable of binding to the LHRH receptor. Preferably, the LHRH analogue, LHRH agonist, LHRH antagonist or combination of LHRH analogue, LHRH agonist or LHRH antagonist is capable of binding to one or more LHRH receptors and are gonadotropin secretory inhibitors or gonadotropin receptor effect blockers.

LHRH agonists that can be used in the present invention can, for example, include the peptides described in Treatment with LHRH analogs: Controversies and perspectives, The Parthenon Publishing Group Ltd. (1996), JP-A-3-503165, JP-A-3-101695, JP-A-7-97334 and JP-A-8-259460 and the like. More specifically, a peptide having the formula:

(Pyr)Glu-R¹-Trp-Ser-R²—R³—R⁴-Arg-Pro-R⁵ (I)

wherein R¹ is His, Tyr, Trp or p-NH₂-Phe; R² is Tyr or Phe; R³ is Gly or D type amino acid residue that may optionally have one or more substituents; R⁴ is Leu, Ile or Nle; and R⁵ is Gly-NH—R⁶ (R⁶ is a hydrogen atom or an alkyl group optionally having a hydroxyl group), NH—R⁷ (R⁷ is a hydrogen atom, an amino group, an alkyl group optionally having a hydroxyl group, or an ureido group (—NH—CO—NH₂)), or a salt thereof, can be used in the present invention.

In the aforementioned formula (I), when R³ is a D type amino acid residue, said D type amino acid can be an α-D-amino acid having up to 9 carbon atoms (i.e., D-Leu, Ile, Nle, Val, Nval, Abu, Phe, Phg, Ser, Thr, Met, Ala, Trp, α-Aibu) or the like. Examples of the substitutents that can be used with R³, include, but are not limited to, tert-butyl, tert-butoxy, tert-butoxycarbonyl, methyl, dimethyl, trimethyl, 2-naphthyl, indolyl-3-yl, 2-methylindolyl, benzyl-imidazo-2-yl and the like. Additionally in formula (I), examples of an alkyl group for R⁶ or R⁷, include, but are not limited to, a C₁₋₄ alkyl group, which is exemplified by methyl, ethyl, propyl, isopropyl, butyl, isobutyl, sec-butyl and tert-butyl.

In addition, a salt of the peptide represented by the formula (I) (which is also referred to as “peptide (I)” herein), include, but are not limited to, an acid salt (i.e., carbonate, bicarbonate, acetate, trifluoroacetate, propionate, succinate etc.) and a metal complex compound (i.e., copper complex, zinc complex etc.) are used. Peptide (I) or a salt thereof can be produced using any method known to those skilled in the art, such as a method described in, for example, U.S. Pat. Nos. 3,853,837, 4,008,209, 3,972,859, GB patent No. 1,423,083, Proceedings of the National Academy of Sciences of the United States of America, vol. 78, pp. 6509-6512 (1981) and the like or a method analogous thereto.

Preferably, peptide (I) can be any one of the following having the below described formulas (a)-(j).

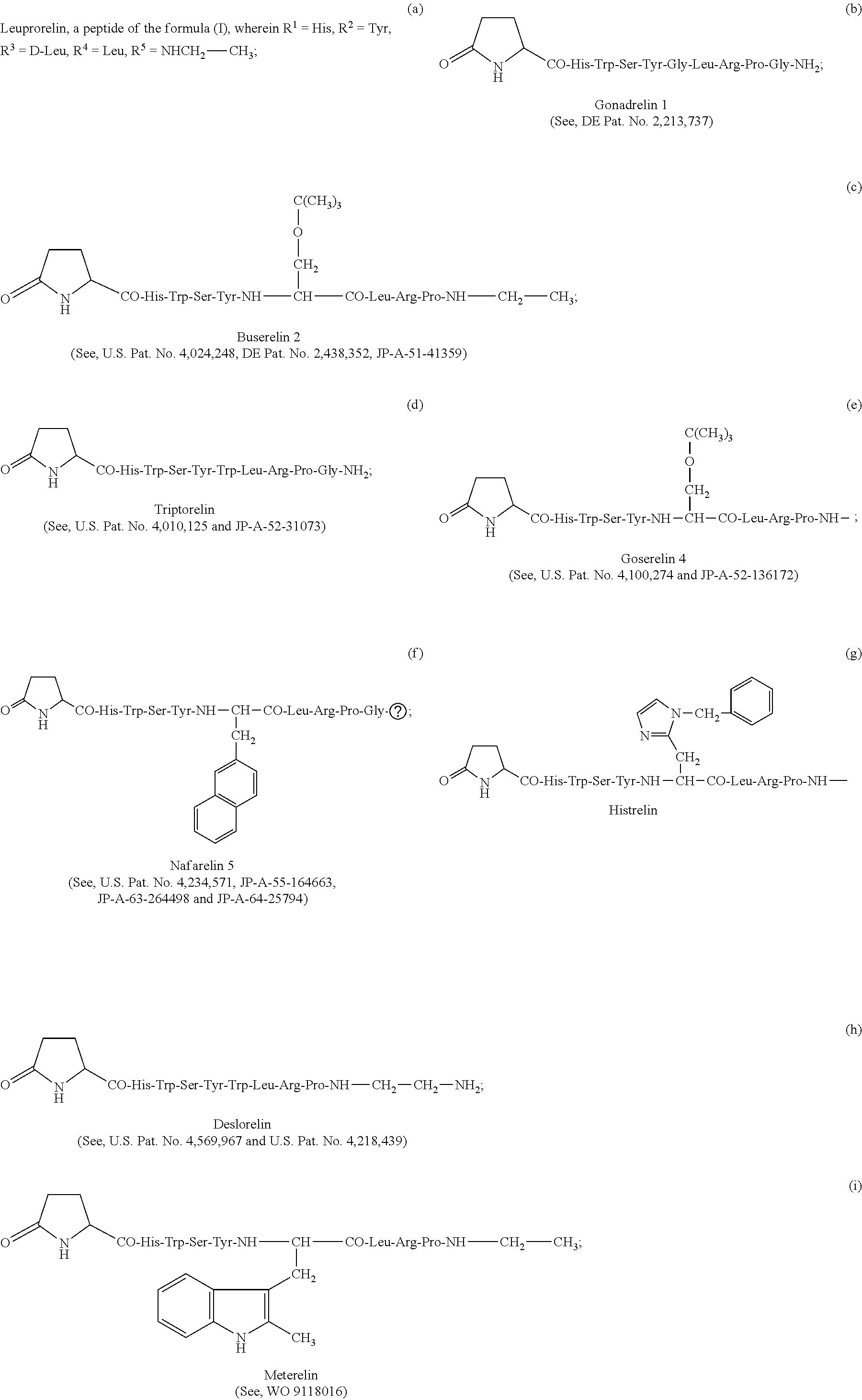

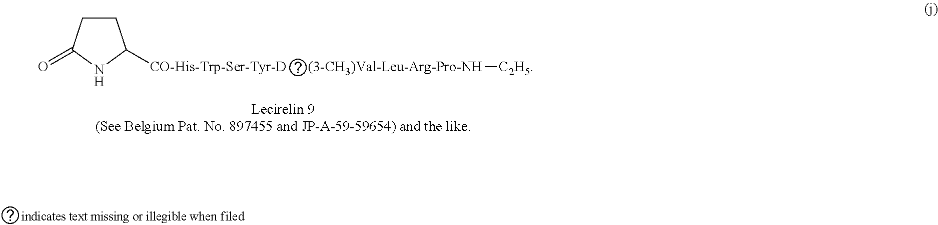

In the aforementioned formulas (c)-(j), an amino acid corresponding to R³ in the formula (I) is in a D-form. The peptide (I) or a salt thereof is preferably leuprorelin or leuprorelin acetate. As used herein, the term “leuprorelin acetate” refers to an acetate of leuprorelin.

LHRH antagonists that can be used in the present invention can, for example, include those disclosed in U.S. Pat. Nos. 4,086,219, 4,124,577, 4,253,997 and 4,317,815, or a peptide represented by the following formula:

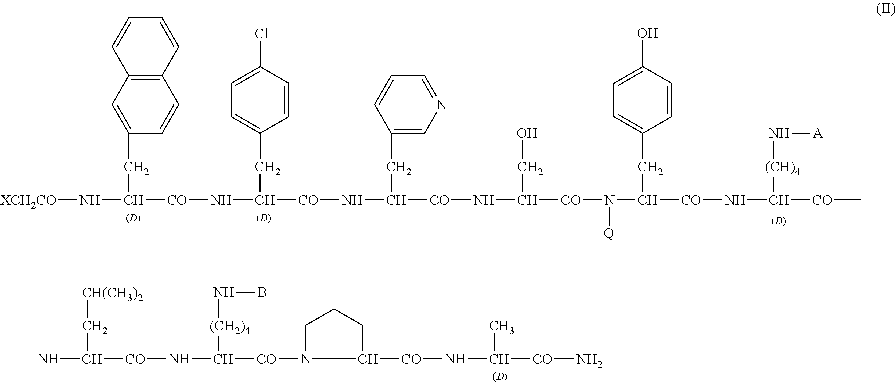

wherein X is a hydrogen or tetrahydrofurylcarboxamide, Q is a hydrogen or methyl, A is nicotinoyl or N,N′-diethylamidino and B is isopropyl or N,N′-diethylamidino (hereinafter is also referred to as “peptide (II)” herein) or a salt thereof. In formula (II), X is preferably tetrahydrofurylcarboxamide, more preferably (2S)-tetrahydrofurylcarboxamide. A is preferably nicotinoyl. B is preferably isopropyl. When peptide (II) has one or more kinds of asymmetric carbon atoms, two or more kinds of optical isomers can be present. Peptide (II) can be used as such optical isomer, or a mixture of these optical isomers.

With respect to a salt of peptide (II), a pharmacologically acceptable salt is preferably used. Examples of such salts, include, but are not limited to, salts of inorganic acids (i.e., hydrochloric acid, sulfuric acid, nitric acid and the like), salts of organic acids (i.e., carbonic acid, bicarbonic acid, succinic acid, acetic acid, propionic acid, trifluoroacetic acid and the like) and the like. Preferably, the salt of peptide (II) is a salt of an organic acid (i.e., carbonic acid, bicarbonic acid, succinic acid, acetic acid, propionic acid, trifluoroacetic acid and the like). Most preferably, the salt of peptide (II) is a salt of acetic acid. More specifically, these salts can be mono, di or tri salts.

More specifically, the peptide (II) or a salt thereof preferably has the following formulas (1)-(4):

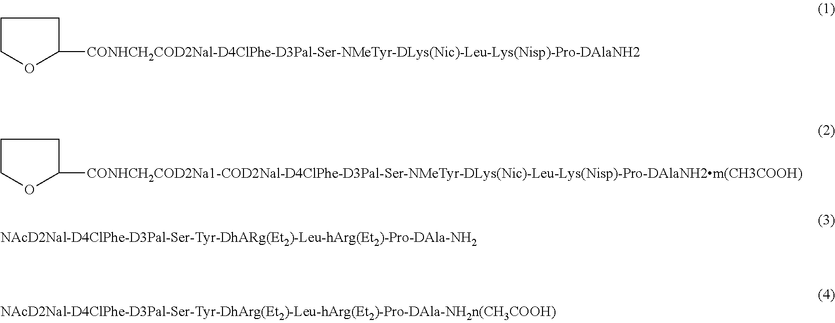

where m is a number of from 1 to 3 and n is a number of from 1 to 3.

The aforementioned formulas (2) and (4) show salts or solvates. Preferably, peptide (II) or a salt thereof has the aforementioned formula (1) or (2), which is particularly preferably an S-isomer.

Peptide (II) or a salt thereof can be produced by any method known to those skilled in the art, such as a method described in JP-A-3-101695 (EP-A 413209), Journal of Medicinal Chemistry, Vol. 35, p. 3942 (1992) and the like, or a method analogous thereto.

Additionally, it is possible to use a linear peptide which is a derivative of LHRH (U.S. Pat. No. 5,140,009 and U.S. Pat. No. 5,171,835), a cyclic hexapeptide derivative (JP-A-61-191698), a bicyclic peptide derivative (Journal of Medicinal Chemistry, Vol. 36, pp. 3265-3273 (1993)) and the like. Examples of non-peptide compounds having an LHRH antagonistic action, compounds described in JP-A-62-116514, WO 95/28405 (JP-A-8-295693), WO 97/14697 (JP-A-9-169767), WO 97/14682 (JP-A-9-169735), WO 96/24597 (JP-A-9-169768), J. Med. Chem., Vol. 32, pp. 2036-2038 (1989) and the like can be used.

Examples of LHRH antagonists that can be used in the present invention include, but are not limited to, abarelix, ganirelix, cetrorelix, 5-(N-benzyl-N-methylaminomethyl)-1-(2,6-difluorobenzyl)-6-[4-(3-methoxyureido)phenyl]-3-phenylthieno[2,3-d]pyrimidine-2,4(1H,3H)-dione, 5-(N-benzyl-N-methylaminomethyl)-1-(2,6-difluorobenzyl)-6-[4-(3-ethylureido)phenyl]-3-phenylthieno[2,3-d]pyrimidine-2,4(1H,3H)-dione and 5-(N-benzyl-N-methylaminomethyl)-1-(2,6-difluorobenzyl)-6-[4-(3-ethylureido)phenyl]-3-phenylthieno[2,3-d]pyrimidine-2,4(1H,3H)-dione hydrochloride.

As used herein, the term “precocious puberty” refers to the appearance of physical signs of puberty, the hormonal signs of puberty and a combination of the physical signs of puberty and hormonal signs of puberty at an earlier age in a subject, preferably a human, than is considered normal. In human girls, precocious puberty is when any of the following develop before eight (8) years of age: breasts, armpit or pubic hair, a rapid height growth (or “growth spurt”), acne, mature external genitalia and/or first menstruation. In human boys, precocious puberty is when any of the following develop before nine (9) years of age: enlarge testes and penis, armpit or pubic hair, a rapid height growth (or “growth spurt”), voice deepening, acne and/or facial hair. Sex steroid levels can be used to determine and diagnose whether a child is suffering from precocious puberty. For example, in boys, total serum testosterone levels can be examined. Methods for determining total serum testosterone levels are known in the art (such as taking a whole blood sample from a subject). Typically, total serum testosterone levels are usually determined in ng/dL. Depending on the reference level used by a laboratory, total serum testosterone levels of 10-30 ng/dL can represent a high level of testosterone that is associated with early or precocious puberty. In girls, estradiol levels can be used to determine and diagnose whether a child is suffering from precocious puberty. Methods for determining estradiol levels are well known in the art (such as taking a whole blood sample from a subject). Estradiol levels are usually determined in pg/mL. Depending on the reference level used by a laboratory, estradiol levels exceeding 20 pg/mL usually represent a high level of estradiol that is associated with early or precocious puberty.

As used herein, the term “subject” refers to an animal, preferably a mammal, including a human or non-human. The terms patient and subject may be used interchangeably herein.

As used herein, the term “test sample” refers to a biological sample obtained from a subject of interest. For example, a test sample can be a biological fluid (e.g., whole blood, serum, plasma, spinal fluid, urine, etc.), a cell sample, or tissue, feces, hair, etc.

The terms “treating” and “treatment” refer to reduction in severity and/or frequency of symptoms, elimination of symptoms and/or underlying cause, prevention of the occurrence of symptoms and/or their underlying cause, and improvement or remediation of damage. Thus, for example, “treating” a patient involves prevention of a particular disorder or adverse physiological event in a susceptible individual as well as treatment of a clinically symptomatic individual by inhibiting or causing regression of a disorder or disease.

### THE PRESENT INVENTION

In one embodiment, the present invention relates to methods for lowering the level of mercury in a subject. More specifically, the methods of the present invention can be used to lower the level of mercury in a subject that has been determined to have a high level of mercury and is diagnosed or suffering from mercury toxicity. Any medical test known to those skilled in the art can be used to determine the level of mercury in a test sample obtained from a subject. The specific type of medical test performed on the test sample is not critical provided that it is capable of determining the level of mercury in a test sample obtained from said subject.

A number of medical tests are known to those skilled in the art for measuring the level of mercury in a subject, particularly a human. For example, one medical test that can be used measures the level of mercury in the whole blood of a subject. Such whole blood tests are well known to those skilled in the art. These blood tests can measure exposure to all three types of mercury (namely, elemental, organic and inorganic mercury). However, because mercury remains in the bloodstream for only a few days after exposure, such a blood test must be done soon after exposure. Typically, non-exposed subjects have mercury levels of 0 to 2 micrograms of mercury per deciliter of blood (μg/dl). Nonetheless, any subject who has a mercury level in his or her whole that is above a laboratory reference level or reference interval (which is known to those skilled in the art to vary from laboratory to laboratory) for mercury is considered to have a “high level of mercury” and thus suffering from mercury toxicity for the purposes of this invention.

Another medical test that can be used to measure the level of mercury in a test sample obtained from a subject is a urine test. Such urine tests are well known to those skilled in the art. Some urine tests measure mercury levels in μg/L and some urine tests measure mercury levels in μg/g creatinine. However, the units used to measure the levels of mercury in a subject are not critical. Regardless of which unit of measurement is used, these urine tests measure exposure to elemental and inorganic mercury. Organic mercury cannot be measured as it is not passed out of the body via urine but rather via the feces. Typically, with urine tests that measure mercury levels in μg/L, non-exposed subjects, who do not suffer from mercury toxicity, frequently have urine mercury levels of 0 to 20 μg/L. However, with urine tests that measure mercury levels in μg/g creatinine, subjects that do not suffer from mercury toxicity, have urine mercury levels less than 3.0 μg/g creatinine. Nonetheless, any subject who has a urine mercury level that is at or above a laboratory reference level for mercury is considered to have a “high level of mercury” and thus suffering from mercury toxicity for the purposes of this invention. Additionally, not only are these urine tests used to determine the levels of mercury in a subject, but these tests can also be used to gauge the efficacy of chelation therapy in a subject.

An additional medical test that can be used to measure the level of body-burden of mercury in a subject is a porphyrin test (potential test sample sources include in the urine, blood and feces of a subject). For example, it has been shown researchers (See Nataf R, Skorupka C, Amet L, Lam A, Springbett A, Lathe R. Porphyrinuria in childhood autistic disorder: implication for environmental toxicity. Toxicol Appl Pharmacol 2006; 214:99-108. Geier D A, Geier M R. A prospective assessment of porphyrins in autistic disorders: a potential marker for heavy metal exposure. Neurotox Res 2006; 10:57-64) that specific porphyrins (precopropophryin, copropophryin and pentacarboxyporphyrin) known to be elevated by an increased body-burden of mercury were significantly elevated in the urine of children with autistic disorders.

In the present invention, for example, a whole blood test, a urine test, a porphyrin test or a combination of a whole blood test and a urine test, a whole blood test and a porphyrin test, or a urine test and a porphyrin test, or a whole blood test, urine test or porphyrin test a can be used to determine the level of mercury in a subject. Based on the results of the medical test, a determination is made by one skilled in the art whether the level of mercury in said subject is high and whether said subject is suffering from mercury toxicity.

Once a determination has been made that a subject has a high level of mercury and is likely suffering from mercury toxicity, the subject can be treated pursuant to the methods of the present invention in order to lower the level of mercury in said subject. More specifically, the methods of the present invention involve administering to said subject a pharmaceutically effective amount of at least one luteinizing hormone releasing hormone composition. Preferably, the at least one luteinizing hormone releasing hormone is a LHRH agonist, such as leuprolide acetate. For example, leuprolide acetate is available as LUPRON® and LUPRON DEPOT® (Takeda Pharmaceutical Company Limited, Osaka, Japan). LUPRON DEPOT® is currently approved and available in adult does of 3.75 mg, 7.5 mg, 11.25 mg, 22.5 and 30 mg and in pediatric doses of 7.5 mg, 11.25 mg and 15 mg dosage forms. LUPRON® is also currently approved and available in adult and pediatric daily doses of 5 mg/ml in 2.8 ml multi-dose vials.

The methods of the present invention involve administering to said subject at least one luteinizing hormone releasing hormone as a LHRH agonist, such as LUPRON® and LUPRON DEPOT®. LUPRON® can be administered in daily doses of about 5 ug/kg per day to about 1.0 mg/kg per day for children (ages 18 years or younger) or about 0.3 to about 5 mg per day to adults. LUPRON DEPOT® can be administered to the subject once at least every 28 days in doses of about 2.5 mg to about 100 mg for adults or about 5 mg to about 100 mg for children. Preferably, LUPRON® is administered in either daily doses of about 20 ug/kg per day to about 150 ug/kg per day for children (ages 18 years or younger). LUPRON® can also be administered at about 0.5 mg to about 10 mg per day to adults. LUPRON DEPOT® is preferably administered to the subject at least once every 28 days in doses of about 5.0 mg to about 75 mg for adults or about 10 mg to about 75 mg for children. Moreover, to achieve the treatment described herein, a subject can be treated with both LUPRON® and LUPRON DEPOT® during the course of the subject's treatment regimen. The LUPRON® and LUPRON DEPOT® can be administered to a subject sequentially, one right after another on the same day, or on different days. For example, LUPRON DEPOT® can be given on day one of treatment along with LUPRON® or LUPRON DEPOT® can be given on day one of treatment and LUPRON® can be given on day three of treatment. Preferably, the LUPRON® is given every day during the course of treatment. Additionally, if necessary, the amount of LUPRON® administered to a subject can be increased in 1.0 mg increments as needed to control the androgen levels and clinical symptoms of the subject. Additionally, the LUPRON DEPOT® can be administered at an additional frequency of more than once every 28 days as needed to control the androgen levels and clinical symptoms of the subject.

Optionally, and if necessary, the subject can also be administered a pharmaceutically effective amount of at least one chelating agent. If a subject is to be administered at least one chelating agent, it is preferred for the purposes of the present invention, the at least one luteinizing hormone releasing hormone composition be administered first to the subject followed by a pharmaceutically effective amount of at least one chelating agent (one the same day or on a different day), or the pharmaceutically effective amount of at least one chelating agent be administered first to the subject followed by a pharmaceutically effective amount of at least one luteinizing hormone releasing hormone (on the same day or on a different day).

Any pharmaceutically acceptable chelating agent can be used. As alluded to above, the chelating agent can be administered to the subject on the same day that the subject is administered the at least one luteinizing hormone releasing hormone composition or the at least one chelating agent can be administered on a different day when the subject is not receiving the at least one luteinizing hormone releasing composition. However, once treatment with a pharmaceutically effective amount of at least one chelating agent treatment has been begun in a subject, administration of the pharmaceutically effective amount at least one chelating agent or treatment to the subject or treatment with the pharmaceutically effective amount at least one chelating agent is continued every day (once or multiple times a day), every other day (once or multiple times a day) or every few days (once or multiple times a day) as needed (i.e., until the level of mercury in the subject has been lowered). In addition, the methods of the present invention contemplate treating a subject with a pharmaceutically effective amount of more than one chelating agent at a time, preferably, as different dosage forms. For example, the present invention contemplates treating a subject with a pharmaceutically effective amount of at least one chelating agent (i.e., a first chelating agent) that is administered transdermally as well as with a pharmaceutically effective amount of at least one chelating agent (i.e., a second chelating agent) that is to be administered orally. Each of these chelating agents (i.e., the first and second chelating agents) can be administered separately, on different days, or on the same day. The treatment with each of these chelating agents (i.e., the first and second chelating agents) can be separate from one another (i.e., the first chelating agent is administered for a period of time and then stopped and treatment with the second chelating agent is begun immediately thereafter), overlap with one another (i.e., the first chelating agent is administered for a period of time and then stopped, but prior to stopping treatment with the first chelating agent, treatment with the second chelating agent is begun), or occur concurrently with one another (i.e., the first and second chelating agents are administered at the same time) and with the administration of the at least one luteinizing releasing hormone composition. The amount of at least one chelating agent to be administered to a subject will vary depending on the chelating agent used and how the chelating agent is to be administered (i.e., such as orally, transdermally, intravenously, orally and transdermally, orally, transdermally and intravenously, orally and intravenously or transdermally and intravenously, etc.). Those skilled in the art will be able to determine the type of chelating agent and amount to be given to a subject. For example, oral DMSA can be given to a child at a dose of from about 2 to about 15 mg/kg and such a dose can be administered to said child up to three times per day. In contrast, transdermal DMPS can be given to a child by applying from about 0.5 to about 5 mg/kg once a day.

Optionally and if necessary, the subject can also be administered a pharmaceutically effective amount of at least one antiandrogenic hormone. Any pharmaceutically acceptable antiandrogenic hormone can be used in the methods of the present invention. The amount of at least one antiandrogenic hormone to be administered to a subject can be from about 50 to about 500 mg per day. The at least one antiandrogenic hormone can be administered to the subject on the same day that the subject is administered the at least one luteinizing hormone releasing hormone composition, at least one chelating agent, at least one luteinizing hormone releasing hormone composition and at least one chelating agent, or on a different day. Additionally, once treatment has begun with the at least one antiandrogenic hormone in a subject, the at least one antiandrogenic hormone can continued to be administered to the subject every day (once or multiple times a day), every other day (once or multiple times a day) or every few days (once or multiple times a day) as needed (i.e., until the level of mercury in the subject has been lowered). The time at which the at least one antiandrogenic hormone is administered to the subject is not critical.

Optionally and if necessary, the subject can also be administered a pharmaceutically effective amount of at least one androgen compound. Any pharmaceutically acceptable androgen compound can be used in the methods of the present invention. The amount of at least one androgen compound to be administered to a subject can be from about 0.1 to about 1,000 mg per day. The at least one androgen compound can be administered to the subject on the same day that the subject is administered the at least one luteinizing hormone releasing hormone composition, at least one chelating agent, the at least one antiandrogenic hormone, at least one luteinizing hormone releasing hormone composition and at least one chelating agent, at least one luteinizing hormone releasing hormone and at least one antiandrogenic hormone, etc., or on a different day. Additionally, once treatment has begun with the at least one androgen compound in a subject, the at least one androgen compound can continued to be administered to the subject every day (once or multiple times a day), every other day (once or multiple times a day) or every few days (once or multiple times a day) as needed (i.e., until the level of mercury in the subject has been lowered). The time at which the at least one androgen compound is administered to the subject is not critical.

Optionally and if necessary, the subject can also be administered vitamin and mineral supplementation. The subject can be administered either a multi-vitamin with minerals or individual vitamins and minerals. Preferably, the subject is given at least 100% of all of the daily recommended vitamins and minerals. Moreover, the vitamin and mineral supplementation can be administered any time during the course of treatment regimen described herein. Preferably, the vitamin and mineral supplementation is administered on days that the subject is not receiving treatment with at least one chelating agent.

Optionally and if necessary, the subject, if a female at or beyond pubertal age can also be administered at least one estrogen compound. The normal age at which puberty occurs in females is between about 10 to about 15 years old. As used herein, the term, “estrogen compound” refers to any substance, natural or synthetic, that exerts a biological or pharmacological action primarily by binding to an estrogen receptor. Estrogen compounds included in this definition are estrogen derivatives, estrogen metabolites, conjugated estrogens, estrogen analogues and estrogen precursors. Also included within this definition are mixtures of more than one estrogen compound. Examples of the at least one estrogen compound that can be used in the present invention, include, but are not limited to:

Conjugated estrogens, natural and synthetic. Natural conjugated estrogens are a mixture of sodium estrone sulfate and sodium equilin sulfate and can be administered daily in the amount of about 0.3 to about 1.25 mg. Synthetic conjugated estrogens can contain up to nine (9) synthetic estrogenic substances. These estrogenic substances can be sodium estrone sulfate, sodium equilin sulfate, sodium 17(alpha)-dihydroequilin sulfate, sodium 17(alpha)-estradiol sulfate, sodium 17(beta)-dihydroequilin sulfate, sodium 17(alpha)-dihydroequilenin sulfate, sodium 17(beta)-dihydroequilenin sulfate, sodium equilenin sulfate and sodium 17(beta)-estradiol sulfate. Synthetic conjugated estrogens can be administered daily in the amount of about 0.3 to about 1.25 mg.

Estradiol acetate. Estradiol acetate can be administered daily in the amount of about 0.45 mg to about 1.8 mg.

Additionally, and optionally, at least one progesterone compound or at least one progestin compound may also be administered with or in combination with the at least one estrogen compound or separately. Examples of the at least one progesterone compound or at least one progestin compound that can be used in the present invention, includes, but are not limited to:

Progesterone. (USP Capsules) Progesterone can be administered daily in a dose of about 100 to about 200 mg orally for 12 days sequentially per 28 day cycle, to women with a uterus who are receiving daily conjugated estrogens tablets.

Norethindrone acetate. Norethindrone acetate can be administered in the form of tablets which can be administered daily in a dose of 2.5 to 10 mg.

The at least one estrogen compound and the at least one progesterone compound can be administered together in combination. An example of at least one estrogen compound and at least one progesterone compound include, but are not limited to:

Estradiol/norethindrone acetate combination tablets (Activella®, Novo Nordisk, Princeton, N. J.) which can be administered daily in a dose of about 1 mg estradiol and about 0.5 mg norethindrone acetate.

It is also preferred that the at least one estrogen compound and optionally, at least one progesterone compound or at least one progestin compound be administered to a child, preferably a female child. It is most preferred that the female child or female adult be at or beyond pubertal age. The normal age at which puberty occurs in females is 10 to 15 years old.

A variety of estrogen compounds, progesterone compound and combinations of estrogen compounds and progesterone compounds are known in the art, commercially available and can be used in the present invention. These include, but are not limited to, Premarin® (Wyeth, Madison, N.J.—Premarin® is conjugated estrogens tablets, USP Premarin® is available as 0.3 mg, 0.45 mg, 0.625 mg, and 0.9 mg tablets), or Activella® which contains 1 mg estradiol and 0.5 mg norethindrone acetate, or Femtrace® (Warner Chilcott, Rockaway, N.J.—Femtrace® are estradiol acetate tablets) for oral administration containing 0.45 mg, 0.9 mg or 1.8 mg estradiol acetate, or synthetic conjugated estrogens, which contain a blend of nine (9) synthetic estrogenic substances. The estrogenic substances are sodium estrone sulfate, sodium equilin sulfate, sodium 17(alpha)-dihydroequilin sulfate, sodium 17(alpha)-estradiol sulfate, sodium 17(beta)-dihydroequilin sulfate, sodium 17(alpha)-dihydroequilenin sulfate, sodium 17(beta)-dihydroequilenin sulfate, sodium equilenin sulfate and sodium 17(beta)-estradiol sulfate. The present invention contemplates that the estrogen compounds and/or progersterone compounds used in the present invention can also consist of various types of birth control pills. Examples of birth control pills that can used include, but are not limited to:

(1) LO/OVRAL (Wyeth, Madison, N.J.) 21 tablets, each containing 0.3 mg of norgestrel (d1-13-beta-ethyl-17-alpha-ethinyl-17-beta-hydroxygon-4-en-3-one), a totally synthetic progestogen, and 0.03 mg of ethinyl estradiol (19-nor-17(alpha)-pregna-1,3,5 (10)-trien-20-yne-3,17-diol), and 7 pink inert tablets;

(2) ORTHO TRI-CYCLEN (Ortho-McNeil Pharmaceutical, Inc., Raritan, N.J.) 28 tablets each containing 0.180 mg of the progestational compound, norgestimate (18,19-Dinor-17-pregn-4-en-20-yn-3-one,17-(acetyloxy)-13-ethyl-,oxime,(17(alpha))-(+)-) and 0.035 mg of the estrogenic compound, ethinyl estradiol (19-nor-17(alpha)-pregna,1,3,5(10)-trien-20-yne-3,17-diol); and

(3) YASMIN (Berlex, Wayne, N.J.) an oral contraceptive regimen consisting of 21 active film coated tablets each containing 3.0 mg of drospirenone and 0.030 mg of ethinyl estradiol and 7 inert film coated tablets.

Once treatment has begun with at least one estrogen compound (and optionally, with at least one progesterone compound or at least one progestin compound) in a subject, the at least one estrogen compound (and optionally, the at least one progesterone compound or at least one progestin compound) can be administered to the subject every day (once or multiple times a day), every other day (once or multiple times a day) as needed (i.e, until the level of mercury in the subject has been lowered). The time at which the at least one estrogen compound (and optionally, the at least one progesterone compound or at least one progestin compound) is administered to the subject is not critical.

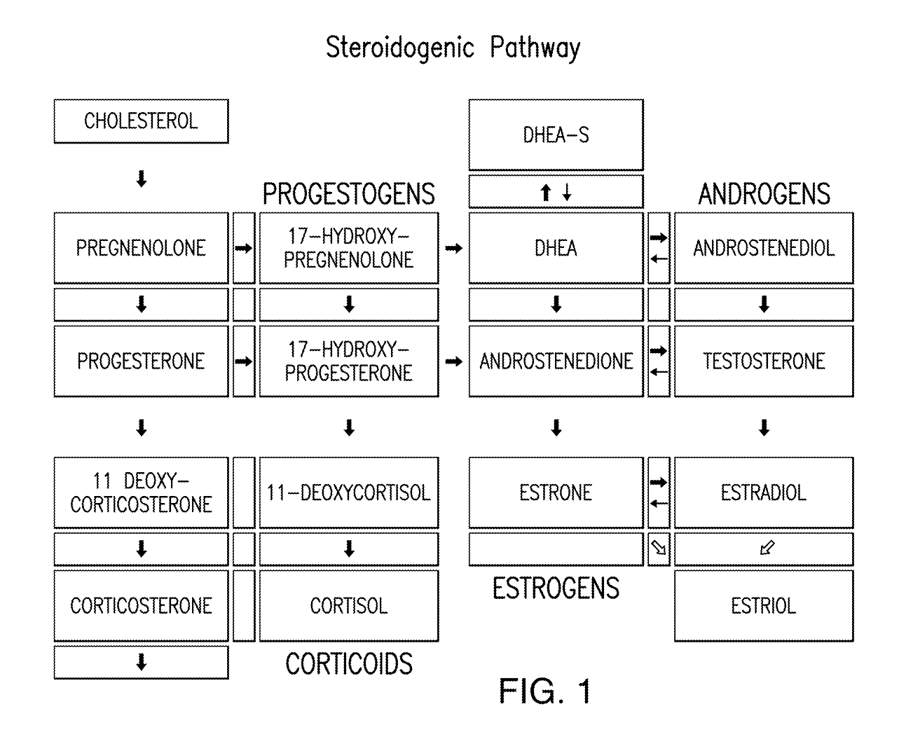

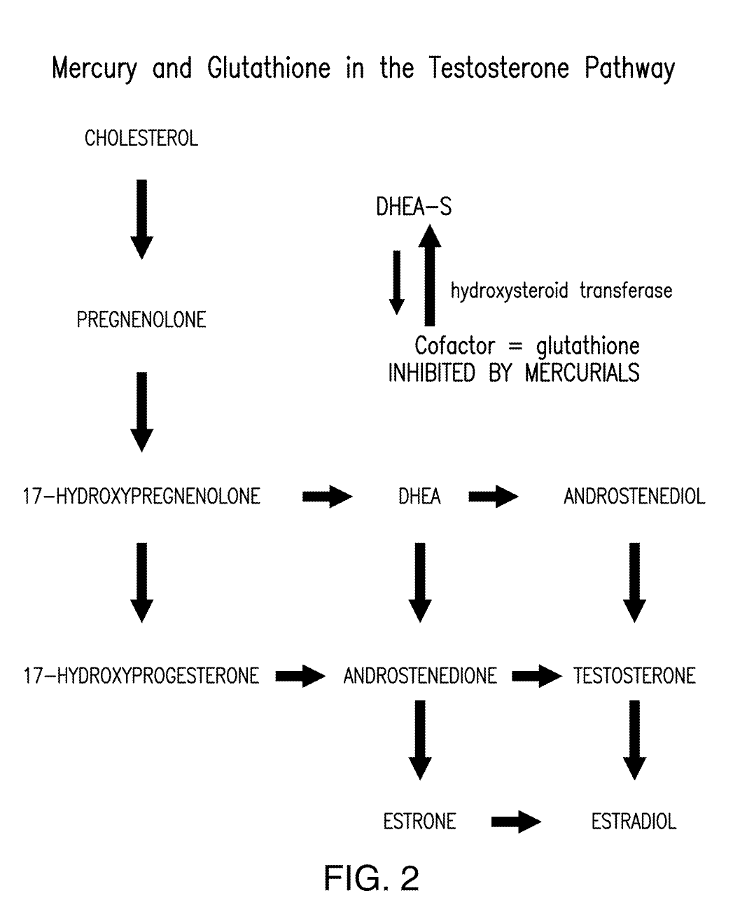

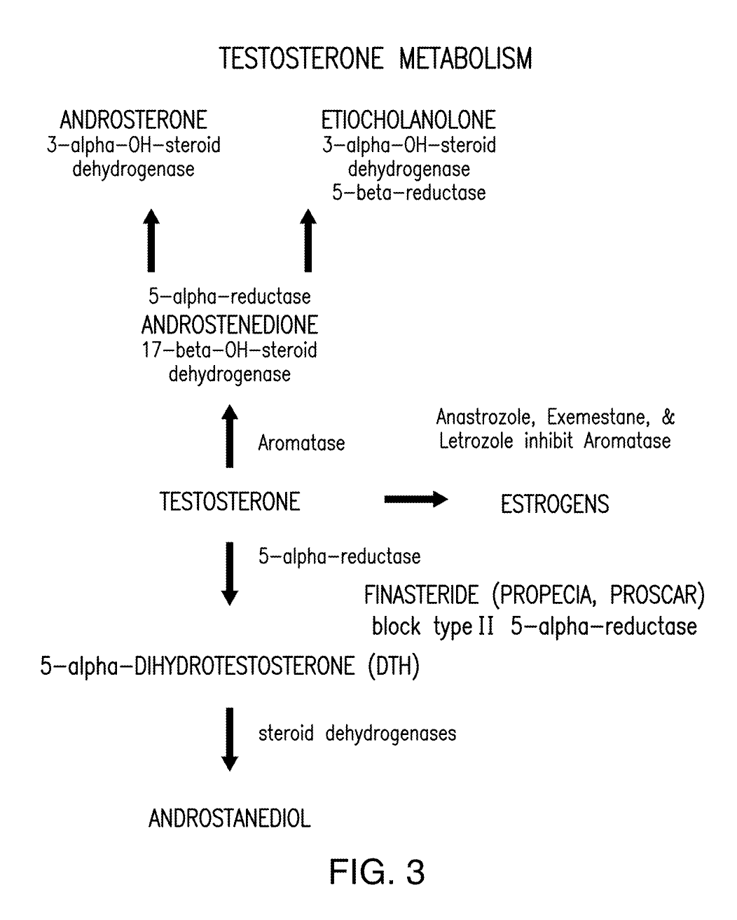

The present inventors have found that many subjects who have been determined to have a high level of mercury (and thus suffer from mercury toxicity) also have low levels of transsulfuration metabolites (Methods for determining the levels of reduced transsulfuration metabolites are well known to those skilled in the art). Sulphation is known to play a role in the testosterone pathway. The testosterone pathway is a part of the steroidogenic pathway (See FIG. 1). More specifically, 3′-phosphoadenosine 5′-phosphosulfate (PAPS) functions as a substrate with hydroxysteroid transferase (HST) in converting DHEA to DHEA-S (See FIG. 2). Most DHEA that is produced in the testosterone synthesis pathway is stored as DHEA-S, thereby reducing the amount that is made into androstrenediol and then eventually into testosterone (See FIGS. 2 and 3).

While not wishing to be bound by any theory, the present inventors believe that when the levels of transsulfuration metabolites in a subject suffering from high levels of mercury are low, HST is inhibited or its level is reduced in its function in converting DHEA to DHEA-S. The result is that the pathway shifts and the amount of testosterone produced in the subject increases. In fact, subjects having a high level of mercury frequently, but not always, also exhibit high levels of one or more androgens, particularly, total serum testosterone. In these instances, as the level of one or more androgens (such as the level of total serum testosterone) in the subject increases, the higher the amount of one or more of said androgens (such as testosterone) is available to bind with mercury. When the one or more androgens bind with mercury, a complex is formed. These androgen-mercury chloride complexes (particularly testosterone-mercury chloride complexes) are difficult to remove from the subject with a chelating agent.

The present inventors have found that the level of mercury in a subject can be lowered by treating a subject with either (a) a pharmaceutically effective amount of at least one luteinizing hormone releasing hormone composition; or (b) a combination of a pharmaceutically effective amount of at least one luteinizing hormone releasing hormone composition and a pharmaceutically effective amount of at least one chelating agent, and then repeating this treatment until the level of mercury in said subjects has been lowered. The at least one luteinizing hormone binds to the LHRH receptor and thus prevents the production of gonadotropins, such as LH and FSH. LH and FSH stimulate the gonads. More specifically, in the testes, LH binds to receptors on Leydig cells, stimulating synthesis and secretion of testosterone. Theca cells in the ovary respond to LH stimulation by secretion of testosterone, which is converted into estrogen by adjacent granulosa cells. By reducing the amount of testosterone being produced in a subject determined to have high levels of mercury, less testosterone is available to bind to mercury. Because few testosterone-mercury complexes are formed, if necessary, more mercury can be removed by administering to the subject at least one chelating agent. Additionally, the pharmaceutically effective amount of at least one luteinizing hormone releasing hormone will lower androgen levels and may raise glutathione levels. The higher glutathione levels may allow for a more effective removal of the mercury and thus indirectly, the use of a pharmaceutically effective amount of at least one luteinizing hormone release hormone may, by itself, also help to lower the body burden of mercury.

The above-described methods (i.e., treatment regimens) are repeated as long as necessary until the level of mercury in the subject is reduced and the patient makes/maintains a significant overall improvement in their symptoms. Optionally, in those subjects who also exhibit elevated levels of one or more androgens, the above-described methods are repeated as long as clinically necessary until the patient makes/maintains a significant overall improvement in their symptoms. Preferably, the aim of such treatment regimens being a significant reduction the level of mercury, and the lowering or reduction and maintenance over time of one or more androgens (such as, but not limited to, the level of total serum testosterone) in the subject to levels well within or below the normal reference range for the patient's age and sex. Methods for determine the levels of one or more androgens are well known to those skilled in the art. Preferably, the level of mercury in the subject is lowered or reduced to a level that is undetectable (using any of the hereinbefore described medical tests). Moreover, if appropriate, the level of one or more androgens (such as, but not limited to, total serum testosterone) is lowered or reduced to a level that is well within the normal range for the patient's age and sex and that these reduced levels of mercury and levels of androgens remain lowered or reduced for a period of at least three months. A determination that the levels of mercury and, optionally, the levels of one or more androgens (such as, but not limited to, total serum testosterone) in a subject has been reduced or lowered can be made by using any medical test, such as a whole blood test or urine test, as described previously herein. The medical test can be performed as many times as necessary in order to determine whether or not the levels of mercury and optionally, the levels of one or more androgens (such as, but not limited to, total serum testosterone) in the subject have been lowered.

As discussed previously herein, at least one antiandrogenic hormone can be optionally administered to a subject. This treatment is administered to a subject because as the testosterone-mercury complexes begin to break apart, there is the potential to release biologically active testosterone into the body. The result is that the released biologically active testosterone may interact at the cellular level with deposit of mercury within cells, and thus produce testosterone-mercury toxicity to such cells. The at least one antiandrogenic hormone administered to a subject can help minimize the functioning of released biologically active testosterone, and hence minimize the potential for testosterone-mercury toxicity to cells within the subject.

The above-described methods can not only be used to treat subject having a high level of mercury (and who suffers from mercury toxicity), but can also be used to treat diseases and disorders that have a mercury component. Such diseases and disorders include, but are not limited to, autism, autism spectrum disorders, attention deficit disorder, attention deficit hyperactivity disorder, mental retardation, Asperger's syndrome, childhood psychoses, stammering, stuttering, tics, repetitive movements, eating disorders, sleep disorders, enuresis, developmental language disorders, developmental speech disorders, developmental delay, Alzheimer's disease, diabetes, heart disease, obesity, amyotrophic lateral sclerosis, nephritic syndrome, renal failure, asthma, systemic lupus, autoimmune thyroiditis, rheumatoid arthritis, arthritis, vasculities, myelitis, glomerulonephritis, optic neuritis, infantile cerebral palsy, epilepsy, schizophrenia, migraine, toxic encephalopathy, cerebral degenerations, anterior horn cell disease, spinocerebellar disease, extrapyramidal disease and myopathy. The present invention also contemplates that subjects having a high level of mercury may also have one or more of the aforementioned diseases or disorders.

In another embodiment, the inventors of the present invention have found that children who have been diagnosed with autism and who also suffer from a high level of mercury (i.e. mercury toxicity) particularly benefit from the methods of the present invention as described herein. Autistic children, male or female, between the ages of two (2) and seventeen (17), particularly benefit from the methods of the present invention. Autistic children who are determined to have a high level of mercury, using any of the hereinbefore described medical tests known to those skilled in the art, can be treated pursuant to the treatment regimens described previously herein. The methods of the present invention have also been found to be useful in treating autistic children who have a high level of mercury and who have also been diagnosed with precocious puberty.

The effectiveness of the above-identified methods in treating children suffering from autism can be monitored or demonstrated though the use of ATEC (Autism, Treatment, Evaluation, Checklist) Form that was developed by the Autism Research Institute (San Diego, Calif.). The ATEC is a one-page form developed by Bernard Rimland and Stephen M. Edelson. It consists of 4 subtests:
1. Speech/Language/Communication (14 items—scores can range from 0-28).
2. Sociability (20 items—scores can range from 0-40).
3. Sensory/Cognitive Awareness (18 items—scores can range from 0-36).
4. Health/Physical/Behavior (25 items—scores can range from 0-75).
   The Autism Research Institute calculates four subscale scores and a total score (total scores can range from 0-180) from the ATEC form. The scores are weighted according to the response and the corresponding subscale. The higher the subscale and total score, the more impaired the subject. The lower the subscale and total score, the less impaired the subject.

In yet another embodiment, the present invention relates to a method of assessing the risk of whether a child is susceptible of developing autism or autism spectrum disorder. More specifically, the inventors have found that children, particularly male children, who have high levels of one or more androgens have a greater risk of developing autism, particularly if these children are exposed to mercury, such as through food, vaccines containing mercury as a preservative, environmental pollution, etc. Therefore, the present invention also a physician to determine, based on a child's androgen level, whether a child would be at risk of developing autism if that child were exposed to mercury.

The method involves first determining the level of one or more androgens of a child, male or female, between the ages of eight (8) months old to eighteen (18) years old by obtaining a test sample from said child. For example, said child could have their level of total serum testosterone examined. Methods for determining the level of total serum testosterone from a test sample are well known in the art. Once the total serum testosterone level of that child has been determined, that level is compared against the reference level for a child of the same age and gender. Reference levels tend to vary depending on the laboratory performing the test. If the child's total serum testosterone level is at the reference level or greater than the reference level for total serum testosterone, then the child is considered to be at risk for developing autism if that child were to be exposed to mercury. Therefore, using this information, a physician could weigh the benefits and risks associated with giving a child with a high total serum testosterone level one or more vaccinations that contains mercury as a preservative. In contrast, if the child's total serum testosterone level is not at the reference level or greater than the reference level for total serum testosterone, then such a child would not be considered to be at risk for developing autism if that child were to be exposed to mercury. For example, at age 8 months, the total serum testosterone level of a male baby is determined to be 8 ng/dL. The reference level of total serum testosterone for a male baby at a similar age at the laboratory is from 1-10 ng/dL. Therefore, given that the child's total serum testosterone level is not at or above the reference level, a determination would be made that this child would be at a low risk of developing autism if exposed to mercury. By way of another example, at age 1 year, the total serum testosterone level of a female baby is determined to be 10 ng/dL. The reference level of total serum testosterone for a female baby at a similar age at the laboratory is from 1-10 ng/dL. Therefore, given that this child's total serum testosterone level is at the reference level, a determination would be made that this child would be at a high risk of developing autism if exposed to mercury. By way of yet another example, at age 18 months, the total serum testosterone level of a male baby is determined to be 11 ng/dL. The reference level of total serum testosterone for a male baby at a similar age at the laboratory is from 1-20 ng/dL. Therefore, given that this child's total serum testosterone level is below the reference level, a determination would be made that this child would not be at risk of developing autism if exposed to mercury. By way of yet another example, at age 2 years, the total serum testosterone level of a male baby is determined to be 27 ng/dL. The reference level of total serum testosterone for a male baby at a similar age at the laboratory is from 1-25 ng/dL. Therefore, given that this child's total serum testosterone level is above the reference level, a determination would be made that this child would be at risk of developing autism if exposed to mercury.

In yet another embodiment, the present invention relates to methods of treating a subject suffering from autism or an autism spectrum disorder. More specifically, the methods of the present invention can be used to treat a subject suffering from autism and wherein said subject also exhibits an elevated level of one or more androgens (including, but not limited to an increase in total serum testosterone or an elevated level of free serum testosterone). In other words, said subjects, in addition to suffering from autism or an autism spectrum disorder, also suffer from hyperandrogenicity. The subjects that treated pursuant to the methods described herein do not have a high level of mercury in their system and thus do not suffer from mercury toxicity. Any medical test known to those skilled in the art can be used to determine the level of one or more androgens (such as, but not limited to, total serum testosterone or the level of free serum testosterone) in a test sample obtained from a subject. The specific type of medical test performed on the test sample is not critical provided that it is capable of determining the level of one or more androgens (such as, but not limited to, the total serum testosterone or the level of free serum testosterone) in a test sample obtained from said subject.

A determination of the severity of autism or an autism spectrum disorder in a subject can be made using the ATEC (Autism, Treatment, Evaluation, Checklist) Form that was developed by the Autism Research Institute (San Diego, Calif.). As mentioned previously herein, the ATEC consists of 4 subtests:
1. Speech/Language/Communication (14 items—scores can range from 0-28).
2. Sociability (20 items—scores can range from 0-40).
3. Sensory/Cognitive Awareness (18 items—scores can range from 0-36).
4. Health/Physical/Behavior (25 items—scores can range from 0-75).

The Autism Research Institute calculates four subscale scores and a total score (total scores can range from 0-180) from the ATEC form. The scores are weighted according to the response and the corresponding subscale. The higher the subscale and total score, the more impaired the subject. The lower the subscale and total score, the less impaired the subject. The ATEC can also be used to monitor the effectiveness of treatment (such as the treatment regimens described herein) of a subject suffering from autism or an autism spectrum disorder.

A number of medical tests are known to those skilled in the art for measuring the level of one or more androgens in a subject, particularly a human. For example, one medical test that can be used measures the level of total serum testosterone or the level of free serum testosterone in the whole blood of a subject. Such whole blood tests are well known to those skilled in the art. As is well known in the art, the total serum testosterone level or the free serum testosterone level of a subject depends on the age and gender of the subject. For example, as discussed in Tietz NW, ed., Clinical Guide to Laboratory Tests, 3^{rd} ed. Philadelphia, Pa.: WB Saunders Co., 1995, 578, the level of free serum testosterone for males between the age of 6-9 years is between 0.01-0.32 ng/dL and for females is between 0.01-0.09 ng/dL, for males between the age of 10-11 years between 0.06-0.57 ng/dL and for females between 0.10-0.52 ng/dL, for males between the age of 12-14 years between 0.14 to 15.60 ng/dL and for females 0.10-0.52 ng/dL, for males between the age of 15-17 years between 8.00-15.90 ng/dL and for females between 0.10-0.52 ng/dL and for male adults between 5.00-21.00 ng/dL and for females 0.10-0.85 ng/dL. Nonetheless, any subject who has a total serum testosterone level that is above a laboratory reference level or reference interval (which is known to those skilled in the art to vary from laboratory to laboratory) is considered to “exhibit an elevated level of total serum testosterone” for the purposes of this invention. Likewise, any subject who has a free serum testosterone level that is above a laboratory reference level or reference interval is considered to “exhibit an elevated level of free serum testosterone”.

In the present invention, for example, a whole blood test, can be used to determine the level of total serum testosterone or free serum testosterone in a subject. Based on the results of the medical test, a determination is made by one skilled in the art whether the level of total serum testosterone or the level of free serum testosterone in said subject is high or elevated.

Once a determination has been made that a subject is suffering from autism or an autism spectrum disorder and further that said subject exhibits an elevated level of one or more androgens, the subject can be treated pursuant to the methods of the present invention in order to lower the level of said one or more androgens in said subject and thus treat the autism or autism spectrum disorder. More specifically, the methods of the present invention involve administering to said subject a pharmaceutically effective amount of at least one luteinizing hormone releasing hormone composition. Preferably, the at least one luteinizing hormone releasing hormone is a LHRH agonist, such as leuprolide acetate. For example, leuprolide acetate is available as LUPRON® and LUPRON DEPOT® (Takeda Pharmaceutical Company Limited, Osaka, Japan). LUPRON DEPOT® is currently approved and available in adult does of 3.75 mg, 7.5 mg, 11.25 mg, 22.5 and 30 mg and in pediatric doses of 7.5 mg, 11.25 mg and 15 mg dosage forms. LUPRON® is also currently approved and available in adult and pediatric daily doses of 5 mg/ml in 2.8 ml multi-dose vials.

The methods of the present invention involve administering to said subject at least one luteinizing hormone releasing hormone as a LHRH agonist, such as LUPRON® and LUPRON DEPOT®. LUPRON® can be administered in daily doses of about 5 ug/kg per day to about 1.0 mg/kg per day for children (ages 18 years or younger) or about 0.3 to about 5 mg per day to adults. LUPRON DEPOT® can be administered to the subject once at least every 28 days in doses of about 2.5 mg to about 100 mg for adults or about 5 mg to about 100 mg for children. Preferably, LUPRON® is administered in either daily doses of about 20 ug/kg per day to about 150 ug/kg per day for children (ages 18 years or younger). LUPRON® can also be administered at about 0.5 mg to about 10 mg per day to adults. LUPRON DEPOT® is preferably administered to the subject at least once every 28 days in doses of about 5.0 mg to about 75 mg for adults or about 10 mg to about 75 mg for children. Moreover, to achieve the treatment described herein, a subject can be treated with both LUPRON® and LUPRON DEPOT® during the course of the subject's treatment regimen. The LUPRON® and LUPRON DEPOT® can be administered to a subject sequentially, one right after another on the same day, or on different days. For example, LUPRON DEPOT® can be given on day one of treatment along with LUPRON® or LUPRON DEPOT® can be given on day one of treatment and LUPRON® can be given on day three of treatment. Preferably, the LUPRON® is given every day during the course of treatment. Additionally, if necessary, the amount of LUPRON® administered to a subject can be increased in 1.0 mg increments as needed to control the androgen levels and clinical symptoms of the subject. The LUPRON DEPOT® can also be administered more than once every 28 days.

Optionally, and if necessary, the subject can also be administered a pharmaceutically effective amount of at least one chelating agent. If a subject is to be administered at least one chelating agent, it is preferred for the purposes of the present invention, the at least one luteinizing hormone releasing hormone composition be administered first to the subject followed by a pharmaceutically effective amount of at least one chelating agent (one the same day or on a different day), or the pharmaceutically effective amount of at least one chelating agent be administered first to the subject followed by a pharmaceutically effective amount of at least one luteinizing hormone releasing hormone (on the same day or on a different day).

Any pharmaceutically acceptable chelating agent can be used. As alluded to above, the chelating agent can be administered to the subject on the same day that the subject is administered the at least one luteinizing hormone releasing hormone composition or the at least one chelating agent can be administered on a different day when the subject is not receiving the at least one luteinizing hormone releasing composition. However, once treatment with a pharmaceutically effective amount of at least one chelating agent treatment has been begun in a subject, administration of the pharmaceutically effective amount at least one chelating agent or treatment to the subject or treatment with the pharmaceutically effective amount at least one chelating agent is continued every day (once or multiple times a day), every other day (once or multiple times a day) or every few days (once or multiple times a day) as needed (i.e., until the level of mercury in the subject has been lowered). In addition, the methods of the present invention contemplate treating a subject with a pharmaceutically effective amount of more than one chelating agent at a time, preferably, as different dosage forms. For example, the present invention contemplates treating a subject with a pharmaceutically effective amount of at least one chelating agent (i.e., a first chelating agent) that is administered transdermally as well as with a pharmaceutically effective amount of at least one chelating agent (i.e., a second chelating agent) that is to be administered orally. Each of these chelating agents (i.e., the first and second chelating agents) can be administered separately, on different days, or on the same day. The treatment with each of these chelating agents (i.e., the first and second chelating agents) can be separate from one another (i.e., the first chelating agent is administered for a period of time and then stopped and treatment with the second chelating agent is begun immediately thereafter), overlap with one another (i.e., the first chelating agent is administered for a period of time and then stopped, but prior to stopping treatment with the first chelating agent, treatment with the second chelating agent is begun), or occur concurrently with one another (i.e., the first and second chelating agents are administered at the same time) and with the administration of the at least one luteinizing releasing hormone composition. The amount of at least one chelating agent to be administered to a subject will vary depending on the chelating agent used and how the chelating agent is to be administered (i.e., such as orally, transdermally, intravenously, orally and transdermally, orally, transdermally and intravenously, orally and intravenously or transdermally and intravenously, etc.). Those skilled in the art will be able to determine the type of chelating agent and amount to be given to a subject. For example, oral DMSA can be given to a child at a dose of from about 2 to about 15 mg/kg and such a dose can be administered to said child up to three times per day. In contrast, transdermal DMPS can be given to a child by applying from about 0.5 to about 5 mg/kg once a day.

Optionally and if necessary, the subject can also be administered a pharmaceutically effective amount of at least one antiandrogenic hormone. Any pharmaceutically acceptable antiandrogenic hormone can be used in the methods of the present invention. The amount of at least one antiandrogenic hormone to be administered to a subject can be from about 50 to about 500 mg per day. The at least one antiandrogenic hormone can be administered to the subject on the same day that the subject is administered the at least one luteinizing hormone releasing hormone composition, at least one chelating agent, at least one luteinizing hormone releasing hormone composition and at least one chelating agent, or on a different day. Additionally, once treatment has begun with the at least one antiandrogenic hormone in a subject, the at least one antiandrogenic hormone can continued to be administered to the subject every day (once or multiple times a day), every other day (once or multiple times a day) or every few days (once or multiple times a day) as needed (i.e., until the level of one or more androgens in the subject has been lowered). The time at which the at least one antiandrogenic hormone is administered to the subject is not critical.

Optionally and if necessary, the subject can also be administered a pharmaceutically effective amount of at least one androgen compound. Any pharmaceutically acceptable androgen compound can be used in the methods of the present invention. The amount of at least one androgen compound to be administered to a subject can be from about 0.1 to about 1,000 mg per day. The at least one androgen compound can be administered to the subject on the same day that the subject is administered the at least one luteinizing hormone releasing hormone composition, at least one chelating agent, the at least one antiandrogenic hormone, at least one luteinizing hormone releasing hormone composition and at least one chelating agent, at least one luteinizing hormone releasing hormone and at least one antiandrogenic hormone, etc., or on a different day. Additionally, once treatment has begun with the at least one androgen compound in a subject, the at least one androgen compound can continued to be administered to the subject every day (once or multiple times a day), every other day (once or multiple times a day) or every few days (once or multiple times a day) as needed (i.e., until the level of one or more androgens in the subject has been lowered). The time at which the at least one androgen compound is administered to the subject is not critical.

Optionally and if necessary, the subject can also be administered vitamin and mineral supplementation. The subject can be administered either a multi-vitamin with minerals or individual vitamins and minerals. Preferably, the subject is given at least 100% of all of the daily recommended vitamins and minerals. Moreover, the vitamin and mineral supplementation can be administered any time during the course of treatment regimen described herein. Preferably, the vitamin and mineral supplementation is administered on days that the subject is not receiving treatment with at least one chelating agent.

Optionally and if necessary, the subject, if a female at or beyond pubertal age can also be administered at least one estrogen compound (using the at least one estrogen compound described previously herein). Additionally, and optionally, at least one progesterone compound or at least one progestin compound may also be administered with or in combination with the at least one estrogen compound. The at least one progesterone compound or at least one progestin compound that can be administered can be the at least one progesterone compound or at least one progestin compound described previously herein. Once treatment has begun with at least one estrogen compound (and optionally, with at least one progesterone compound or at least one progestin compound) in a subject, the at least one estrogen compound (and optionally, the at least one progesterone compound or at least one progestin compound) can be administered to the subject every day (once or multiple times a day), every other day (once or multiple times a day) as needed (i.e, until the level of one or more androgens in the subject has been lowered). The time at which the at least one estrogen compound (and optionally, the at least one progesterone compound or at least one progestin compound) is administered to the subject is not critical.

The present inventors have found that in many subjects who have been determined to have elevated levels of one or more androgens also have low levels of transsulfuration metabolites (methods for determining the levels of reduced transsulfuration metabolites are well known to those skilled in the art). Sulphation is known to play a role in the testosterone pathway. The testosterone pathway is a part of the steroidogenic pathway (See FIG. 1). More specifically, PAPS functions as a substrate with hydroxysteroid transferase (“HST”) in converting DHEA to DHEA-S (See FIG. 2). Most DHEA that is produced in the testosterone synthesis pathway is stored as DHEA-S, thereby reducing the amount that is made into androstrenediol and then eventually into testosterone (See FIGS. 2 and 3).

While not wishing to be bound by any theory, the present inventors believe that when the levels of transsulfuration metabolites in a subject suffering from autism are low, HST is inhibited or its level is reduced in its function in converting DHEA to DHEA-S. The result is that the pathway shifts and the amount of testosterone produced in the subject increases. In fact, subjects suffering from autism or autism spectrum disorders frequently are determined to have high levels of total serum testosterone.

The present inventors have found that the level of one or more androgens in a subject suffering from autism or autism spectrum disorders (but who is not suffering from mercury toxicity) can be lowered by treating a subject with either (a) a pharmaceutically effective amount of at least one luteinizing hormone releasing hormone composition; or (b) a combination of a pharmaceutically effective amount of at least one luteinizing hormone releasing hormone composition and a pharmaceutically effective amount of at least one chelating agent, and then repeating this treatment until the level of at least one of said androgens in said subjects has been lowered. The at least one luteinizing hormone binds to the LHRH receptor and thus prevents the production of gonadotropins, such as LH and FSH. LH and FSH stimulate the gonads. More specifically, in the testes, LH binds to receptors on Leydig cells, stimulating synthesis and secretion of testosterone. Theca cells in the ovary respond to LH stimulation by secretion of testosterone, which is converted into estrogen by adjacent granulosa cells.

The above-described methods (i.e., treatment regimens) are repeated as long as clinically necessary to improve/maintain a significant overall improvement in the patient's symptoms. Preferably, the aim of such treatment regimens is to lower or reduce and maintain over time the level of said at least one androgen to a level that is well within or below the normal range for the patient's age and sex. A determination that the level of said at least one androgen in a subject has been reduced or lowered can be made by using any medical test, such as a whole blood test, as described previously herein. The medical test can be performed as many times as necessary in order to determine whether or not the level of said at least one androgen in the subject has been lowered.

As discussed previously herein, at least one antiandrogenic hormone can be optionally administered to a subject. This treatment is administered to a subject because if there is any biologically active testosterone in the body, the at least one antiandrogenic hormone administered to a subject can help minimize the functioning of released biologically active testosterone, and hence minimize the potential for testosterone effects on the subject.

The above-described methods can not only be used to treat subject suffering from autism or autism spectrum disorders, but can also be used to treat diseases and disorders that have a elevated levels of one or more androgens. Such diseases and disorders include, but are not limited to, autism, autism spectrum disorders, attention deficit disorder, attention deficit hyperactivity disorder, mental retardation, Asperger's syndrome, childhood psychoses, stammering, stuttering, tics, repetitive movements, eating disorders, sleep disorders, enuresis, developmental language disorders, developmental speech disorders, developmental delay, Alzheimer's disease, diabetes, heart disease, obesity, amyotrophic lateral sclerosis, nephritic syndrome, renal failure, asthma, systemic lupus, autoimmune thyroiditis, rheumatoid arthritis, arthritis, vasculities, myelitis, glomerulonephritis, optic neuritis, infantile cerebral palsy, epilepsy, schizophrenia, migraine, toxic encephalopathy, cerebral degenerations, anterior horn cell disease, spinocerebellar disease, extrapyramidal disease and myopathy. The present invention also contemplates that subjects having an elevated level of one or more androgens may also have one or more of the aforementioned diseases or disorders.

In still another embodiment, the present invention relates to methods of treating a subject suffering from autism or an autism spectrum disorder. The subjects treated pursuant to the methods described herein do not exhibit: (1) a high level of mercury in their system and thus do not suffer from mercury toxicity; or (2) a high level of at least one androgen and thus do not suffer from hyperandrogenicity.

A determination of the severity of autism or an autism spectrum disorder in a subject can be made using the ATEC (Autism, Treatment, Evaluation, Checklist) Form that was developed by the Autism Research Institute (San Diego, Calif.). As mentioned previously herein, the ATEC consists of 4 subtests:
1. Speech/Language/Communication (14 items—scores can range from 0-28).
2. Sociability (20 items—scores can range from 0-40).
3. Sensory/Cognitive Awareness (18 items—scores can range from 0-36).
4. Health/Physical/Behavior (25 items—scores can range from 0-75).

The Autism Research Institute calculates four subscale scores and a total score (total scores can range from 0-180) from the ATEC form. The scores are weighted according to the response and the corresponding subscale. The higher the subscale and total score, the more impaired the subject. The lower the subscale and total score, the less impaired the subject. The ATEC can also be used to monitor the effectiveness of treatment (such as the treatment regimens described herein) of a subject suffering from autism or an autism spectrum disorder.

Once a determination has been made that a subject is suffering from autism or an autism spectrum disorder, the subject can be treated pursuant to the methods of the present invention in order to lower the level of one or more androgens in said subject and thus treat the autism or autism spectrum disorder. More specifically, the methods of the present invention involve administering to said subject a pharmaceutically effective amount of at least one luteinizing hormone releasing hormone composition. Preferably, the at least one luteinizing hormone releasing hormone is a LHRH agonist, such as leuprolide acetate. For example, leuprolide acetate is available as LUPRON® and LUPRON DEPOT® (Takeda Pharmaceutical Company Limited, Osaka, Japan). LUPRON DEPOT® is currently approved and available in adult does of 3.75 mg, 7.5 mg, 11.25 mg, 22.5 and 30 mg and in pediatric doses of 7.5 mg, 11.25 mg and 15 mg dosage forms. LUPRON® is also currently approved and available in adult and pediatric daily doses of 5 mg/ml in 2.8 ml multi-dose vials.

The methods of the present invention involve administering to said subject at least one luteinizing hormone releasing hormone as a LHRH agonist, such as LUPRON® and LUPRON DEPOT®. LUPRON® can be administered in daily doses of about 5 ug/kg per day to about 1.0 mg/kg per day for children (ages 18 years or younger) or about 0.3 to about 5 mg per day to adults. LUPRON DEPOT® can be administered to the subject once at least every 28 days in doses of about 2.5 mg to about 100 mg for adults or about 5 mg to about 100 mg for children. Preferably, LUPRON® is administered in either daily doses of about 20 ug/kg per day to about 150 ug/kg per day for children (ages 18 years or younger). LUPRON® can also be administered at about 0.5 mg to about 10 mg per day to adults. LUPRON DEPOT® is preferably administered to the subject at least once every 28 days in doses of about 5.0 mg to about 75 mg for adults or about 10 mg to about 75 mg for children. Moreover, to achieve the treatment described herein, a subject can be treated with both LUPRON® and LUPRON DEPOT® during the course of the subject's treatment regimen. The LUPRON® and LUPRON DEPOT® can be administered to a subject sequentially, one right after another on the same day, or on different days. For example, LUPRON DEPOT® can be given on day one of treatment along with LUPRON® or LUPRON DEPOT® can be given on day one of treatment and LUPRON® can be given on day three of treatment. Preferably, the LUPRON® is given every day during the course of treatment. Additionally, if necessary, the amount of LUPRON® administered to a subject can be increased in 1.0 mg increments as needed to control the androgen levels and clinical symptoms of the subject. The LUPRON DEPOT® can also be administered more than once every 28 days.

Optionally, and if necessary, the subject can also be administered a pharmaceutically effective amount of at least one chelating agent. If a subject is to be administered at least one chelating agent, it is preferred for the purposes of the present invention, the at least one luteinizing hormone releasing hormone composition be administered first to the subject followed by a pharmaceutically effective amount of at least one chelating agent (one the same day or on a different day), or the pharmaceutically effective amount of at least one chelating agent be administered first to the subject followed by a pharmaceutically effective amount of at least one luteinizing hormone releasing hormone (on the same day or on a different day).

Any pharmaceutically acceptable chelating agent can be used. As alluded to above, the chelating agent can be administered to the subject on the same day that the subject is administered the at least one luteinizing hormone releasing hormone composition or the at least one chelating agent can be administered on a different day when the subject is not receiving the at least one luteinizing hormone releasing composition. However, once treatment with a pharmaceutically effective amount of at least one chelating agent treatment has been begun in a subject, administration of the pharmaceutically effective amount at least one chelating agent or treatment to the subject or treatment with the pharmaceutically effective amount at least one chelating agent is continued every day (once or multiple times a day), every other day (once or multiple times a day) or every few days (once or multiple times a day) as needed (i.e., until the level of mercury in the subject has been lowered). In addition, the methods of the present invention contemplate treating a subject with a pharmaceutically effective amount of more than one chelating agent at a time, preferably, as different dosage forms. For example, the present invention contemplates treating a subject with a pharmaceutically effective amount of at least one chelating agent (i.e., a first chelating agent) that is administered transdermally as well as with a pharmaceutically effective amount of at least one chelating agent (i.e., a second chelating agent) that is to be administered orally. Each of these chelating agents (i.e., the first and second chelating agents) can be administered separately, on different days, or on the same day. The treatment with each of these chelating agents (i.e., the first and second chelating agents) can be separate from one another (i.e., the first chelating agent is administered for a period of time and then stopped and treatment with the second chelating agent is begun immediately thereafter), overlap with one another (i.e., the first chelating agent is administered for a period of time and then stopped, but prior to stopping treatment with the first chelating agent, treatment with the second chelating agent is begun), or occur concurrently with one another (i.e., the first and second chelating agents are administered at the same time) and with the administration of the at least one luteinizing releasing hormone composition. The amount of at least one chelating agent to be administered to a subject will vary depending on the chelating agent used and how the chelating agent is to be administered (i.e., such as orally, transdermally, intravenously, orally and transdermally, orally, transdermally and intravenously, orally and intravenously or transdermally and intravenously, etc.). Those skilled in the art will be able to determine the type of chelating agent and amount to be given to a subject. For example, oral DMSA can be given to a child at a dose of from about 2 to about 15 mg/kg and such a dose can be administered to said child up to three times per day. In contrast, transdermal DMPS can be given to a child by applying from about 0.5 to about 5 mg/kg once a day.

Optionally and if necessary, the subject can also be administered a pharmaceutically effective amount of at least one antiandrogenic hormone. Any pharmaceutically acceptable antiandrogenic hormone can be used in the methods of the present invention. The amount of at least one antiandrogenic hormone to be administered to a subject can be from about 50 to about 500 mg per day. The at least one antiandrogenic hormone can be administered to the subject on the same day that the subject is administered the at least one luteinizing hormone releasing hormone composition, at least one chelating agent, at least one luteinizing hormone releasing hormone composition and at least one chelating agent, or on a different day. Additionally, once treatment has begun with the at least one antiandrogenic hormone in a subject, the at least one antiandrogenic hormone can continued to be administered to the subject every day (once or multiple times a day), every other day (once or multiple times a day) or every few days (once or multiple times a day) as needed (i.e., until the level of one or more androgens in the subject has been lowered). The time at which the at least one antiandrogenic hormone is administered to the subject is not critical.

Optionally and if necessary, the subject can also be administered a pharmaceutically effective amount of at least one androgen compound. Any pharmaceutically acceptable androgen compound can be used in the methods of the present invention. The amount of at least one androgen compound to be administered to a subject can be from about 0.1 to about 1,000 mg per day. The at least one androgen compound can be administered to the subject on the same day that the subject is administered the at least one luteinizing hormone releasing hormone composition, at least one chelating agent, the at least one antiandrogenic hormone, at least one luteinizing hormone releasing hormone composition and at least one chelating agent, at least one luteinizing hormone releasing hormone and at least one antiandrogenic hormone, etc., or on a different day. Additionally, once treatment has begun with the at least one androgen compound in a subject, the at least one androgen compound can continued to be administered to the subject every day (once or multiple times a day), every other day (once or multiple times a day) or every few days (once or multiple times a day) as needed (i.e., until the level of one or more androgens in the subject has been lowered). The time at which the at least one androgen compound is administered to the subject is not critical.

Optionally and if necessary, the subject can also be administered vitamin and mineral supplementation. The subject can be administered either a multi-vitamin with minerals or individual vitamins and minerals. Preferably, the subject is given at least 100% of all of the daily recommended vitamins and minerals. Moreover, the vitamin and mineral supplementation can be administered any time during the course of treatment regimen described herein. Preferably, the vitamin and mineral supplementation is administered on days that the subject is not receiving treatment with at least one chelating agent.

Optionally and if necessary, the subject, if a female at or beyond pubertal age can also be administered at least one estrogen compound (using the at least one estrogen compound described previously herein). Additionally, and optionally, at least one progesterone compound or at least one progestin compound may also be administered with or in combination with the at least one estrogen compound. The at least one progesterone compound or at least one progestin compound that can be administered can be the at least one progesterone compound or at least one progestin compound described previously herein. Once treatment has begun with at least one estrogen compound (and optionally, with at least one progesterone compound or at least one progestin compound) in a subject, the at least one estrogen compound (and optionally, the at least one progesterone compound or at least one progestin compound) can be administered to the subject every day (once or multiple times a day), every other day (once or multiple times a day) as needed (i.e, until the level of one or more androgens in the subject has been lowered). The time at which the at least one estrogen compound (and optionally, the at least one progesterone compound or at least one progestin compound) is administered to the subject is not critical.

The present inventors have found that the levels of one or more androgens in a subject suffering from autism or autism spectrum disorders can be lowered by treating a subject with either (a) a pharmaceutically effective amount of at least one luteinizing hormone releasing hormone composition; or (b) a combination of a pharmaceutically effective amount of at least one luteinizing hormone releasing hormone composition and a pharmaceutically effective amount of at least one chelating agent, and then repeating this treatment until the level of at least one androgen in said subjects has been lowered and maintained. The at least one luteinizing hormone binds to the LHRH receptor and thus prevents the production of gonadotropins, such as LH and FSH. LH and FSH stimulate the gonads. More specifically, in the testes, LH binds to receptors on Leydig cells, stimulating synthesis and secretion of testosterone. Theca cells in the ovary respond to LH stimulation by secretion of testosterone, which is converted into estrogen by adjacent granulosa cells.

The above-described methods (i.e., treatment regimens) are repeated as long as necessary to significantly improve/maintain an overall improvement in the patient's symptoms. Preferably, the aim of such treatment regimens is to lower or reduce and maintain over time the at least one said androgen to a level that is well within or below the normal range for the patient's age and sex. A determination that the level of at least one androgen in a subject has been reduced or lowered can be made by using any medical test, such as a whole blood test, as described previously herein. The medical test can be performed as many times as necessary in order to determine whether or not the androgen in the subject has been lowered.

At least one antiandrogenic hormone can optionally be administered to a subject. This treatment is administered to a subject because if there is any biologically active testosterone in the body, the at least one antiandrogenic hormone administered to a subject can help minimize the functioning of released biologically active testosterone, and hence minimize the potential for testosterone effects on the subject.

The above-described methods can not only be used to treat subject suffering from autism or autism spectrum disorders, but can also be used to treat diseases and disorders. Such diseases and disorders include, but are not limited to, autism, autism spectrum disorders, attention deficit disorder, attention deficit hyperactivity disorder, mental retardation, Asperger's syndrome, childhood psychoses, stammering, stuttering, tics, repetitive movements, eating disorders, sleep disorders, enuresis, developmental language disorders, developmental speech disorders, developmental delay, Alzheimer's disease, diabetes, heart disease, obesity, amyotrophic lateral sclerosis, nephritic syndrome, renal failure, asthma, systemic lupus, autoimmune thyroiditis, rheumatoid arthritis, arthritis, vasculities, myelitis, glomerulonephritis, optic neuritis, infantile cerebral palsy, epilepsy, schizophrenia, migraine, toxic encephalopathy, cerebral degenerations, anterior horn cell disease, spinocerebellar disease, extrapyramidal disease and myopathy.

Now by way of example, and not of limitation, examples of the present invention shall now be given.

### Example 1

### Autistic Child X

The patient was an eight year old white male who was born in 1996 and diagnosed with autism (said child is hereinafter referred to as “Child X”). Child X was the product of a full term spontaneous vaginal delivery. Child X had good APGAR (Activity, Pulse, Grimace (reflex irritability), Appearance, Respiration) scores and was believed to be totally normal at birth. Child X developed normally meeting all of his developmental milestones during his first year of life. In addition, Child X had all of his childhood vaccines in keeping with the recommended childhood vaccine schedule. Specifically, at 28 weeks gestation the mother of Child X was administered a Rhₒ immune globulin with approximately 70 micrograms of mercury. Moreover, from birth to approximately 15 months, Child X received 150 micrograms of mercury from his childhood vaccines. During his second year of life, Child X lost his language skills and declined into a fully autistic state. More specifically, Child X developed severe gastrointestinal problems that are often seen in autistic children. In fact, Child X never passed a normally formed stool. Child X's disorder fit into what is now commonly labeled as “regressive autism”.

From Oct. 21, 2000 through Feb. 3, 2002, Child X was treated with dimercaptosuccinic acid (DMSA) and spilled toxic levels of mercury in his urine. During this time, Child X was able to pedal his tricycle, his focus and attention was better and he attempted to say words. He was sleeping well and his bowel habits were better. In addition, his appetite was good and he was interacting more and exhibiting more outward expression. In fact, Child X began using scissors.

On Nov. 5, 2000, Child X's urine was collected and examined for toxic metals, specifically, aluminum, antimony, arsenic, beryllium, bismuth, cadmium, lead, mercury, nickel, platinum, thallium, thorium, tin, tungsten and uranium. The toxic metals were reported as μg/g creatinine in order to account for urine dilution variations. Child X's creatinine was 43.4 mg/dL. The results for the toxic metals found in Child X's urine are shown below in Table 1.

TABLE 1

| | Results | |
|---|---|---|
| Toxic Metals | μg/g creatinine | Reference Range |
| Aluminum | 26 | <35 |
| Antimony | 0.4 | <5 |
| Arsenic | 98 | <100 |
| Beryllium | <dl* | <0.5 |
| Bismuth | 0.8 | <30 |
| Cadmium | 1.4 | <2 |
| Lead | 12 | <15 |
| Mercury | 15 | <3 |
| Nickel | 21 | <12 |
| Platinum | 0.2 | <2 |
| Thallium | 0.2 | <14 |
| Thorium | <dl | <12 |
| Tin | 5.5 | <6 |
| Tungsten | <dl | <23 |
| Uranium | <dl | <1 |

*<dl = less than detection limit

As shown in Table 1 above, the level of mercury in Child X's urine was elevated.

On Sep. 30, 2001, Child X's urine was collected and examined for toxic metals, specifically, aluminum, antimony, arsenic, beryllium, bismuth, cadmium, lead, mercury, nickel, platinum, thallium, thorium, tin, tungsten and uranium. The toxic metals were reported as μg/g creatinine in order to account for urine dilution variations. Child X's creatinine was 54 mg/dL. The results for the toxic metals found in Child X's urine are shown below in Table 2.

TABLE 2

| | Results | |
|---|---|---|
| Toxic Metals | μg/g creatinine | Reference Range |
| Aluminum | 14 | <35 |
| Antimony | <dl* | <5 |
| Arsenic | 37 | <100 |
| Beryllium | <dl | <0.5 |
| Bismuth | <dl | <30 |
| Cadmium | 0.5 | <2 |
| Lead | 8.6 | <15 |
| Mercury | 5.4 | <3 |
| Nickel | 11 | <12 |
| Platinum | <dl | <2 |
| Thallium | 0.2 | <14 |
| Thorium | <dl | <12 |
| Tin | 1.6 | <6 |
| Tungsten | 0.3 | <23 |
| Uranium | <dl | <1 |

*<dl = less than detection limit

As shown in Table 2 above, the level of mercury in Child X's urine was elevated. As a result of this test a diagnosis of heavy metal toxicity, specifically, mercury toxicity, was made.

While Child X did exhibit some improvement as a result of the DMSA treatment, Child X continued to be in special class at school and received intensive speech and other behavior therapy. Child X was still unable to speak any words and could not even point to his own body parts. Child X followed verbal commands poorly, if at all, and still would only minimally interact with his peers. His behavior problems had become increasingly intolerable with age. For example, on one occasion, he severely bit his father.

Child X's prior medical work up had included chromosomes indicating that he was a 46, XY without consistent structural or numerical chromosome anomalies, a negative DNA screen for fragile-X, a negative DNA screen for Rett Syndrome and a negative newborn screen for genetic disorders. Screening for serum amino acid levels, thyroid function abnormalities and urine for reducing substances were all negative. Child X's family history is completely negative for autism or any other neurological disorders. In fact, both of his parents have advanced degrees.

On Oct. 23, 2004, Child X's blood was drawn and a laboratory work-up performed. Child X's total serum testosterone was determined to be 25 ng/dL. The reference level of total serum testosterone for a male child of Child X's age at this laboratory was from 0-25 ng/dL. Therefore, Child X's total serum testosterone was determined to be at the high end of the reference level. It was also noted that he exhibited clinical signs of precocious puberty including increased body hair and sexual masturbatory behavior. Therefore, a diagnosis of precious puberty was made. Child X had normal CBC, liver, and kidney function testing.

On Nov. 23, 2004, prior to the initiation of therapy in Child X, the severity of autistic symptoms in Child X were assessed using an ATEC (Autism, Treatment, Evaluation, Checklist) Form developed by the Autism Research Institute (San Diego, Calif.). It was observed that Child X overall had severe autistic symptoms placing him in the 90-99 percentile of severity, with Child X being most profoundly affected in the areas of sociability and sensory/cognitive awareness placing him in the 90-99 percentile of severity.

On Nov. 24, 2004, Child X was given a single shot of LUPRON DEPOT® (leuprolide acetate, Takeda Pharmaceutical Company Limited, Osaka, Japan) in the amount of 22.5 mg. No observable side effects were noted. Within a few days, Child X's behavior and attentiveness were noted to be markedly improved. On Dec. 1, 2004, transdermal DMPS treatment was begun on Child X. Specifically, Child X received a 1.5 mg transdermal DMPS dose/kg bodyweight every other day. After initiation of this chelation therapy, Child X was observed to become somewhat more hyperactive but this soon stabilized as he adjusted to the therapeutic regimen. On Dec. 3, 2004, Child X's total serum testosterone level was tested and determined to be 17 ng/dL. On Jan. 11, 2005, the total serum testosterone level of Child X was again tested and determined to be 19 ng/dL. Because of the improvement exhibited by Child X and the absence of observable side effects, Child X was given a second shot of LUPRON DEPOT® (22.5 mg) on Jan. 20, 2005. On Jan. 22, 2005, oral DMSA treatment was begun. Specifically, Child X received 7.5 mg DMSA/kg bodyweight three times per day of the oral DMSA every other day, on the days when he was also being administered transdermal DMPS (1.5 mg transdermal DMPS dose/kg bodyweight). On Jan. 28, 2005, the total serum testosterone level of Child X was tested and determined to be 32 ng/dL. On Feb. 10, 2005, the total serum testosterone level of Child X was again tested and determined to be 25 ng/dL. On Feb. 19, 2005, treatment was begun with cyproterone acetate (Androcur (Schering A G, Germany)). Specifically, Child X received 50 mg tablets three times per day. On Feb. 28, 2005, the total serum testosterone level of Child X was again tested and determined to be less than 10 ng/dL. On Mar. 18, 2005, the total serum testosterone level of Child X was again tested and determined to be 20 ng/dL. On Mar. 25, 2005, Child X was given a third shot of LUPRON DEPOT® (22.5 mg). On Apr. 8, 2005, the total serum testosterone level of Child X was tested and determined to be 10 ng/dL. On May 5, 2005, the total serum testosterone level of Child X was tested and determined to be less than 10 ng/dL. On May 25, 2005, Child X was given a fourth shot of LUPRON DEPOT® (22.5 mg). On Jun. 28, 2005, the total serum testosterone level of Child X was tested and determined to be 20 ng/dL. On Jul. 14, 2005, Child X was given a fifth shot of LUPRON DEPOT® (22.5 mg). On Aug. 15, 2005, the total serum testosterone level of Child X was tested and determined to be 23 ng/dL.

Child X was assessed by laboratory work-up for biochemical and genomic susceptibility factors to mercury toxicity. On Aug. 15, 2005, Child X's blood was drawn and a laboratory work-up performed. Child X's serum homocysteine was determined to be 5.0 micromoles/L. The reference level of serum homocysteine for a male child of Child X's age at this laboratory was 5.10-13.9 micromoles/L. On Aug. 18, 2005, Child X's blood was drawn again and another laboratory work-up performed. Child X's plasma cysteine was 2.72 mg/dL, plasma sulfate was 2.90 mg/dL, and reduced glutathione was 20 mg/dL. The reference levels for each of these tests for a male child of Child X's age at this laboratory were 3.10-3.90 mg/dL for plasma cysteine, 2.90 mg/dL for plasma sulfate and ≧32 mg/dL for reduced glutathione, respectively.

Within days of the first shot of LUPRON DEPOT® on Nov. 24, 2004, Child X's gastrointestinal symptoms were markedly improved. More specifically, Child X produced normal stools for the first time in seven years. A remarkable improvement in his behavior, attentiveness and mentation were also observed within a few days of the first LUPRON DEPOT® shot. Child X was able to point to most of his body parts accurately and he began to try to imitate speech sounds. Child X's ability to follow verbal commands improved markedly and he began to interact with his siblings and peers. Within a few days of the second shot of LUPRON DEPOT®, Child X learned to swing by himself using leg timing for propulsion. Prior to receiving any of the shots of LUPRON DEPOT®, Child X could not even stay on the swing when pushed by others. Child X also began to be able to feed himself and his attention span and interest for the first time allowed him to watch and be interested in television shows. Child X began to play interactively with toys that he had never done previously. Child X also began, for the first time, to say “no” and to specifically ask for items that he wanted. Child X continues to improve on a daily basis in his mentation, attempts at speech, his interaction with others and his environment. Child X's bowel problems seem to be cured in that he continues to form normal stools. Child X continues to rapidly progress in his behavior and learning.

The improvement in Child X's mentation has been quantitatively documented. It was observed that Child X's Individualized Report Card I for the school year prior to initiation of therapy (namely, the 2003-04 school year) demonstrated that Child X had not mastered any skills in the areas of self help, general knowledge, language, social and emotional development, motor development and enrichment activities. Subsequently, it was observed that Child X's Individualized Report Card I for the mid reporting of the school year while receiving the above described therapy (namely, the 2004-05 school year) demonstrated that Child X had mastered skills in the areas of self help (uses eating utensils appropriately; washes hands independently; takes care of own toileting), general knowledge (observes likenesses and differences in objects and pictures; classifies objects according to color and shape; has left/right orientation), language (follows oral directions), social and emotional development (cooperates in group activities; accepts adult guidance; accepts consequences of own behavior; demonstrates adequate self-control; follows school rules; respects rights and property of others; demonstrates good manners) and motor development (traces simple lines; runs; jumps; hops; throws a ball). It was then observed that Child X's Individualized Report Card I for the end reporting of the school year while receiving the above described therapy (namely, the 2004-05 school year) demonstrated that Child X had mastered skills in the areas of self help (uses eating utensils appropriately; taking off and putting on his outer garments; washes hands independently; takes care of own toileting), general knowledge (recognizes and names body parts; recognizes name in print; writes name from memory; observes likenesses and differences in objects and pictures; classifies objects according to color, size and shape; has left/right orientation), language (states his full name; initiates greetings and farewells; responds to greetings and farewells; asks for assistance when necessary; speaks in short phrases; uses simple sentences; follows oral directions; attends to the speaker), social and emotional development (cooperates in group activities; accepts adult guidance; accepts consequences of own behavior; demonstrates adequate self-control; follows school rules; respects rights and property of others; demonstrates good manners; attempts new tasks in a positive manner) motor development (traces simple lines; traces name; copies name; runs; jumps; hops; catches a ball; throws a ball) and enrichment activities (participates in group singing; responds to rhythms and music; participates in activities; participates in food preparation activities). Additionally, on Jul. 30, 2005, an ATEC Form was used to evaluate the severity of autistic symptoms in Child X (therapy treatment day 248). It was observed that Child X had shown significant overall improvement from the previous ATEC form evaluation conducted on Nov. 23, 2004. Specifically it was observed that Child had improved on the ATEC form from the 90-99 percentile of autistic severity on Nov. 23, 2004 to the 20-29 percentile of autistic severity on Jul. 30, 2005. It was observed that Child X had shown the most significant improvements in the areas of sociability (90-99 percentile of autistic severity on Nov. 23, 2004 to the 20-29 percentile of autistic severity on Jul. 30, 2005) and sensory/cognitive awareness (90-99 percentile of autistic severity on Nov. 23, 2004 to the 20-29 percentile of autistic severity on Jul. 30, 2005).

### Example 2

### Autistic Child Y

The patient was a six year old white male who was born in 1999 and diagnosed with autism (said child is hereinafter referred to as “Child Y”). Child Y was the product of a full term spontaneous vaginal delivery. Child Y had good APGAR scores and was believed to be totally normal at birth. Child Y developed normally meeting all of his developmental milestones during his first year of life. In addition, Child Y had all of his childhood vaccines in keeping with the recommended childhood vaccine schedule. Specifically, from birth to 18 months of age, Child Y had received 137.5 micrograms of mercury from his childhood vaccines. By the end of his second year of life, Child Y lost all of his language skills and declined into a fully autistic state. More specifically, Child Y developed severe gastrointestinal problems that are often seen in autistic children. Child Y never passed a normally formed stool. In fact, Child Y had an endoscopy on Jun. 23, 2003 which showed terminal ileal lymphonodular hyperplasia and inflammatory nodules of the rectosigmoid. The ileal pathology was confirmed on biopsy, and the remainder of the colon appeared to be normal. The upper endoscopy was impressive in that streaking nodular distal esophagitis was noted grossly and confirmed histologically. Child Y's disorder fit into what is now commonly labeled as “regressive autism”.

From Jun. 26, 2002 to May 2, 2003, Child Y was treated with DMSA and spilled toxic levels of mercury in his urine. During this time, Child Y did not show significant improvement in his autism.

On Jun. 29, 2002, Child Y's urine was collected and examined for toxic metals, specifically, aluminum, antimony, arsenic, beryllium, bismuth, cadmium, lead, mercury, nickel, platinum, thallium, thorium, tin, tungsten and uranium. The toxic metals were reported as μg/g creatinine in order to account for urine dilution variations. Child Y's creatinine was 6.9 mg/dL. The results for the toxic metals found in Child Y's urine are shown below in Table 3.

TABLE 3

| | Results | |
|---|---|---|
| Toxic Metals | μg/g creatinine | Reference Range |
| Aluminum | <dl* | <35 |
| Antimony | <dl | <5 |
| Arsenic | 31 | <100 |
| Beryllium | <dl | <0.5 |
| Bismuth | <dl | <30 |
| Cadmium | <dl | <2 |
| Lead | <dl | <15 |
| Mercury | 29 | <3 |
| Nickel | <dl | <12 |
| Platinum | <dl | <2 |
| Thallium | 0.9 | <14 |
| Thorium | <dl | <12 |
| Tin | 5.1 | <6 |
| Tungsten | <dl | <23 |
| Uranium | <dl | <1 |

*<dl = less than detection limit

As shown in Table 3 above, the level of mercury in Child Y's urine was elevated. On Jul. 15, 2002, a diagnosis of heavy metal toxicity, specifically, mercury toxicity, was made.

On Dec. 24, 2002, Child Y's urine was again collected and examined for toxic metals, specifically, aluminum, antimony, arsenic, beryllium, bismuth, cadmium, lead, mercury, nickel, platinum, thallium, thorium, tin, tungsten and uranium. The toxic metals were reported as μg/g creatinine in order to account for urine dilution variations. Child Y's creatinine was 8.6 mg/dL. The results for the toxic metals found in Child Y's urine are shown below in Table 4.

TABLE 4

| | Results | |
|---|---|---|
| Toxic Metals | μg/g creatinine | Reference Range |
| Aluminum | <dl* | <35 |
| Antimony | <dl | <5 |
| Arsenic | 23 | <100 |
| Beryllium | <dl | <0.5 |
| Bismuth | <dl | <30 |
| Cadmium | 1.6 | <2 |
| Lead | <dl | <15 |
| Mercury | 11 | <3 |
| Nickel | <dl | <12 |
| Platinum | <dl | <2 |
| Thallium | 0.6 | <14 |
| Thorium | <dl | <12 |
| Tin | 3.3 | <6 |
| Tungsten | 1.6 | <23 |
| Uranium | <dl | <1 |

*<dl = less than detection limit

As shown in Table 4 above, the level of mercury in Child Y's urine was still very elevated.

Child Y was assessed by laboratory work-up for biochemical and genomic susceptibility factors to mercury toxicity. On Jan. 20, 2003, Child Y's blood was drawn and a laboratory work-up performed. Child Y's plasma cysteine was determined to be 2.58 mg/dL. The reference level of plasma cysteine for a male child of Child Y's age at this laboratory was 3.10-3.90 mg/dL. On Jun. 2, 2004, Child Y's blood was drawn and a laboratory work-up performed. Child Y's reduced glutathione was determined to be 20 mg/dl. The reference level of reduced glutathione for a male child of Child Y's age at this laboratory was ≧32 mg/dL, respectively. In addition, a genomic survey was performed on Nov. 3, 2003 on Child Y which demonstrated that Child Y had SNPs in the MTHFR gene.

On Jan. 29, 2005, Child Y's blood was drawn and a laboratory work-up performed. Child Y's total serum testosterone was determined to be 20 ng/dL. The reference level of total serum testosterone for a male child of Child Y's age at this laboratory was from 0-20 ng/dL. Therefore, Child Y's total serum testosterone was determined to be at the high end of the reference level. It was also noted that he exhibited clinical signs of precocious puberty including increased body hair and genital development. Therefore, a diagnosis of precious puberty was made. Child Y had normal CBC, liver, and kidney function testing.

On Apr. 1, 2005, prior to the initiation of therapy in Child Y, the severity of autistic symptoms in Child Y were assessed using the ATEC Form. It was observed that Child Y overall had severe autistic symptoms placing him in the 70-79 percentile of severity, with Child Y being most profoundly affected in the area of sensory/cognitive awareness placing him in the 90-99 percentile of severity.

On Apr. 2, 2005, Child Y was given a single shot of LUPRON DEPOTS (leuprolide acetate, Takeda Pharmaceutical Company Limited, Osaka, Japan) in the amount of 22.5 mg. No observable side effects were noted. Within a few days, Child Y's gastrointestinal symptoms began to improve and he began to have well formed normal stools, which he had not had previously. In addition, Child Y's behavior and attentiveness were noted to be markedly improved. In addition, improvement in Child Y's receptive and expressive language skills has been noted. Child Y is trying to say more words and is repeating more words. On Apr. 5, 2005, transdermal DMPS treatment was begun on Child Y. Specifically, Child Y received a 1.5 mg transdermal DMPS dose/kg bodyweight every other day. On Apr. 16, 2005, the total serum testosterone level of Child Y was tested and determined to be 48 ng/dL. On Apr. 30, 2005, the total serum testosterone level of Child Y was again tested and determined to be 12 ng/dL. On May 14, 2005, the total serum testosterone level of Child Y was again tested and determined to be less than 10 ng/dL. On May 21, 2005, Child Y was given a second shot of LUPRON DEPOT® (22.5 mg). On May 22, 2005, treatment was begun with cyproterone acetate (Androcur (Schering A G, Germany)). Specifically, Child Y received 50 mg tablets three times per day. On May 23, 2005, oral DMSA treatment was begun. Specifically, Child Y received 7.5 mg DMSA/kg bodyweight three times per day of the oral DMSA every other day, on the days when he was also being administered transdermal DMPS (1.5 mg transdermal DMPS dose/kg bodyweight). On Jun. 18, 2005, the total serum testosterone level of Child Y was again tested and determined to be 17 ng/dL. On Jul. 2, 2005, the total serum testosterone level of Child Y was again tested and determined to be 16 ng/dL. On Jul. 9, 2005, Child Y was given a third shot of LUPRON DEPOT® (22.5 mg). On Jul. 16, 2005, the total serum testosterone level of Child Y was again tested and determined to be 15 ng/dL. On Jul. 30, 2005, the total serum testosterone level of Child Y was again tested and determined to be 13 ng/dL.

It has been observed as the therapy has progressed that Child Y has begun to show skills that were not apparent prior to the initiation of therapy. Child Y has begun to visually recognize and verbally call for his mother use appropriate expressive language skills. It has also been observed that Child Y has begun to visually recognize, communicate, and interact (showing increasing levels of affection) with other members of the family.

Specifically, in quantitative terms, an ATEC form on May 30, 2005 was used to evaluate the severity of autistic symptoms in Child Y (therapy treatment day 58). It was observed that Child Y showed an overall improvement with the most significant improvement in the area of speech/language/communication (80-89 percentile of autistic severity on Apr. 1, 2005 to 60-69 percentile of autistic severity on May 30, 2005).

Subsequently, Child Y's teaching assistant has specifically documented the following newly acquired skills for Child Y observed for period from Jul. 18, 2005 through Aug. 26, 2005:
“Child Y hardly ever initiates requested actions and seldom words. However, I have found that he knows a lot. If I offer him my arm, he will use it as a pointer. This is hardly foolproof and not all results are positive as he does not always focus. If he is wandering in his mind, his choices are not correct. If I tell him to focus and he does, his use of my arm feels much more purposeful and is highly accurate for the tasks currently presented to him. We began doing this the end of July. In the interim, it has become clear that he knows all the letters of the alphabet, both upper case and lower case. He also can read digits at least to 100. He can read many words. We spread flash cards with words in front of him and asked him to “show me”: ‘horse’ for example. He has seen these cards many times in the past. He can match animals to their pictures and color cubes to color mats. Also shapes. I have just begun using my hand under Child Y's to see if he would write. This is harder as his touch is light and I have to hold the pen. Nevertheless, I am sure that Child Y can both recognize and spell his name. When I ask him to write his name, I must tell him his first and last name. He goes right to it and has completed it four times. The third day I also asked him to write one plus one equals two and followed with a one for the next problem. He then said ‘+2=3’. I asked him after that to do the next one without specifying what to write and he wrote ‘1+3=4’. We did not use equations, but instead wrote in columns. Today, I asked him to write a three word sentence and he wrote it. I did not spell any of the words for him. (Please note that I am not at all sure that this is free from my influence, but I am sure that Child Y knows a great deal and can read quite well.)
He is beginning to take charge of the writing when we write with my hand over his and he is holding the pen. We have been writing this way for several months. With respect to words: Child Y seems to be using words a little more readily recently. Also, if I ask him what a pig says, he may provide an incorrect ‘sound’, but it will be an animal sound. If I ask him for a color, he may provide an incorrect color, but it will be a color. Child Y also seems to be able to recognize clocks showing the hour and the half hour for any ‘hour’. He knows how to read a digital time. He also can respond correctly to ‘Show me: quarter, dime, nickel, penny’ (Show me commands require his use of my hand).”

### Example 3

### Autistic Child A

The patient, child A, was a six year old white male who was seen by a physician for work up and possible treatment of a neurodevelopmental disorder of unknown origin.

Child A was the product of a full term, uneventful pregnancy born to parents both of whom had no medical problems other than allergies. Child A was born by c-section with no complications and his neonatal course was completely uneventful. He met all of his developmental milestones, both physical and mental, for his first year and a half of life. His development slowed by 18 months of age and he underwent regression from 24 to 30 months of age. He was diagnosed with autism at age two and a half by his attending pediatrician. Child A has suffered from constipation and diarrhea since the age of thirteen months. Child A was reported to have suffered from problems with sleep for many years that resulted in Child A not be being able to completely sleep through the night.

Child A had been previously tested and found to be negative for Fragile X, chromosomal abnormalities, and plasma amino acid abnormalities. Additionally, the Child A had a head MRI that was normal.

Child A was evaluated using an Autism Treatment Evaluation Checklist (ATEC) which showed significant overall impairments (50-59^{th} percentile of severity), significant impairments in his speech/language/communications skills (80-89^{th} percentile of severity), sensory cognitive awareness skills (60-69^{th} percentile of severity), health/physical/behavior skills (60-69^{th} percentile of severity) and sociability skills (0-9^{th} percentile of severity).

Child A also presented with signs and symptoms of premature puberty, including: hair growth (hair on his back, legs, and penis), genital development and early sexual behaviors, significant muscle development, significant growth spurt (97 percentile of height), and occasional aggressive behaviors.

Laboratory analyses for androgen metabolites revealed an elevated serum total testosterone=23 ng/dL (age- and sex-adjusted LabCorp reference range=0-20 ng/dL) and an above average level of serum/plasma DHEA=62 ng/dL (age- and sex-adjusted LabCorp reference range=29-66 ng/dL).

On the bases of his physical and laboratory findings, a diagnosis of premature puberty was made. After extensive discussions with his parents concerning the risks, benefits, and alternative treatments available, a decision was made to place Child A on a course of LUPRON® therapy.

The therapy was begun with an initial intramuscular injection (IM) of Pediatric LUPRON DEPOT® (leuprolide acetate, Takeda Pharmaceutical Company Limited, Osaka, Japan) 15 mg followed immediately by daily subcutaneous injections of 0.2 ml of daily LUPRON® (which was 1 mg per injection which with a body weight of about 60 pounds is approximately 50 micrograms per kilogram, (actually 55 micrograms per kilogram)). The daily dose of LUPRON® was gradually increased in 0.1 ml increments as indicated by clinical and laboratory monitoring to a final dose of 0.4 ml per day (which is 83 micrograms per kilogram).

With the advent of his LUPRON® therapy, Child A's serum testosterone decline to a less than detectable level, namely, less than 20 ng/dL (Quest Laboratories). Additionally, Child A's DHEA declined to 30 ng/dL.

Within several days of the first LUPRON® administration, Child A's parents reported a marked normalization of all of his gastrointestinal (GI) functions (namely, no more diarrhea or constipation). Subsequently, they also reported major improvements in Child A's attention, cognitive awareness, sleep patterns and receptive language skills. Child A's educators observed greater compliance with tasks, and improved cognitive performance. The observed improvements using LUPRON® have been to seen to remain stable or even to exhibit further improvements in the areas of attention, cognitive awareness, and receptive language skills.

### Example 4

### Autistic Child B

The patient, Child B, was a seven year old white female who was seen by a physician for work up and possible treatment of a neurodevelopmental disorder of unknown origin. Child B was the product of a full term, uneventful pregnancy. Her mother at the time of her pregnancy had no diagnosed medical problems. Child B's father was in good health except that he had been diagnosed with encephalitis as a child. Child B was born by vaginal delivery with induction due to failure to progress. There were no birthing complications and patient's neonatal course was completely uneventful. Child B met all of her developmental milestones, both physical and mental, for her first year of life but was observed to regress at 14 months of age. Child B was diagnosed with autism spectrum disorder at age eighteen months by her attending physician. Gastro-intestinal problems also were observed to begin at 14 months of age consisting primarily of diarrhea.

Laboratory testing of the Child B was negative for Fragile X, chromosomal abnormalities, plasma amino acid abnormalities, Rett syndrome, Angleman/Prader-Willi syndrome, and subtelomere chromosomal anomalies.

Child B was evaluated using an ATEC which showed significant overall impairments (80-89^{th} percentile of severity), significant impairments in her speech/language/communications skills (70-79^{th} percentile of severity), sensory cognitive awareness skills (70-79^{th} percentile of severity), and health/physical/behavior skills (80-89^{th} percentile of severity), and sociability skills (90-99^{th} percentile of severity).

Child B also presented with signs and symptoms of premature puberty, including: hair growth (hair on her legs, and face), and early sexual behaviors.

Laboratory analyses for androgen metabolites revealed an elevated serum testosterone=18 ng/dL (age- and sex-adjusted LabCorp reference range=0-10 ng/dL) and a significantly elevated level of serum/plasma DHEA=202 ng/dL (age- and sex-adjusted LabCorp reference range=73-165 ng/dL).

On the bases of her physical and laboratory findings, a diagnosis of premature puberty was made. After extensive discussions with her parents concerning the risks, benefits, and alternative treatments available, a decision was made to place Child B on a course of LUPRON® therapy.

The therapy was begun with daily subcutaneous injections of 0.3 ml of LUPRON® (which was 1.5 mg per injection which with a body weight of about 60 pounds is approximately 50 micrograms per kilogram, (actually 55 micrograms per kilogram)). This daily dose was increased by the addition of an a IM injection of Pediatric LUPRON DEPOT® 15 mg as indicated by clinical and laboratory monitoring to a final dose of 2.0 mg per day (which is 74 micrograms per kilogram).

With the advent of her LUPRON® therapy, Child B's serum testosterone declined to less than detectable (less than 10 ng/dL). Her DHEA declined to 144 ng/dL. Her parents reported major improvements in attention, cognitive awareness, and receptive language skills. However, Child B's LUPRON® therapy was interrupted for several weeks due to a lack of drug supply. During this period, Child B was observed to regress to a condition approaching her former level of impairment. When the LUPRON® therapy was re-initiated the observed improvements on the LUPRON® therapy returned and have been to seen to remain stable or even to exhibit further improvements in attention, cognitive awareness and receptive language skills.

### Example 5

### Young Adult C

The patient, Young Adult C, was an eighteen year old white male who was seen by a physician for work up and possible treatment of a neurodevelopmental disorder of unknown origin. Young Adult C was the product of a full term uneventful pregnancy. The patient's parents are both in good health. The patient was born by full term spontaneous vaginal delivery. There were no birthing complications and patient's neonatal course was completely uneventful. He met all of his developmental milestones, both physical and mental, for his first year of life but was observed to regress at 18 months of age.

Laboratory testing of Young Adult C was negative for Fragile X, chromosomal abnormalities, plasma amino acid abnormalities, Rett syndrome, Angleman/Prader-Willi syndrome, and subtelomere chromosomal anomalies. Additionally, Young Adult C had a head MRI, the results of which were normal. Young Adult C was diagnosed with autism spectrum disorder at age three and a half by his attending physician.

The patient was evaluated using an ATEC which showed significant overall impairments (80-89^{th} percentile of severity), significant impairments in his speech/language/communications skills (30-39^{th} percentile of severity), sensory cognitive awareness skills (40-49^{th} percentile of severity), health/physical/behavior skills (90-99^{th} percentile of severity) and sociability skills (70-79^{th} percentile of severity).

Young Adult C also presented with extreme aggressive behaviors including being destructive, violent, and was reported to hit and injure himself and others.

Laboratory analyses for androgen metabolites revealed an elevated serum free testosterone=23.03 ng/dL (age- and sex-adjusted LabCorp reference range=5.00-21.00 ng/dL), a significantly elevated level of serum LH=8.7 ng/dL (age- and sex-adjusted LabCorp reference range=0.5-5.3 ng/dL) and a significantly elevated percent free testosterone level of 4.12 (age- and sex-adjusted LabCorp reference range=1.00 to 2.70). Young Adult C was found to have a serum testosterone=559 ng/dL (age- and sex-adjusted LabCorp reference range=241-827 ng/dL).

On the bases of his physical and laboratory findings and after extensive discussions with his parents concerning the risks, benefits, and alternative treatments available, a decision was made to place Young Adult C on a course of LUPRON® therapy.

The therapy was begun an IM injection of Pediatric LUPRON DEPOT® 15 mg. This was augmented with 0.2 ml of daily LUPRON® injected subcutaneously. This daily dose was gradually increased in 0.1 ml increments as indicated by clinical and laboratory monitoring to a dose of 0.5 ml per day (since Young Adult C weighs approximately 145 pounds (66 Kg) is 45 micrograms per kilogram).

With the advent of his LUPRON® therapy, Young Adult C's serum testosterone declined (namely, to 28 ng/dL). His serum free testosterone declined to 0.59 ng/dL and his percent free testosterone declined to 2.09.

The patient was re-evaluated after 156 days on LUPRON® therapy using the ATEC which then showed significant improvements as follows: overall impairments (30-39^{th} percentile of severity, down from 80-89^{th} percentile of severity), impairments in his speech/language/communications skills (30-39^{th} percentile of severity representing no change from the previous level), sensory cognitive awareness skills (30-39^{th} percentile of severity down from 40-49^{th} percentile of severity), health/physical/behavior skills (70-79^{th} percentile of severity down from 90-99^{th} percentile of severity) and sociability skills (50-59^{th} percentile of severity down from 70-79^{th} percentile of severity). His parents and his educators reported major improvements in attention, cognitive awareness, receptive language skills and especially in a reduced level of aggressive behaviors. It was observed that the reduction in the patient's aggressive behaviors resulted in a reduction of self-mutation and physical violence towards others. Young Adult C still suffers from mood swings and occasional sleep problems.

### Example 6

### Child D

The patient, Child D, was an eleven year old white male who was seen by a physician for work up and possible treatment of a neurodevelopmental disorder of unknown origin. Child D was the product of a full term uneventful pregnancy. The patient's parents were both in good health at the time of Child's D delivery, except for the father having insulin dependent diabetes. The patient was born by full term repeat C-section delivery. There were no significant birthing complications and patient's neonatal course was completely uneventful. Child D met all of his developmental milestones, both physical and mental, for his first year of life but was observed to regress at 12-14 months of age.

Laboratory testing of Child D was negative for Fragile X, chromosomal abnormalities, plasma amino acid abnormalities, and subtelomere chromosomal anomalies. Additionally, Child D had a Wood's Lamp examination that was normal. Child D was diagnosed with autism by his attending physician.

The patient was evaluated using an ATEC which showed significant overall impairments (80-89^{th} percentile of severity), significant impairments in his speech/language/communications skills (70-79^{th} percentile of severity), sensory cognitive awareness skills (50-59^{th} percentile of severity), health/physical/behavior skills (80-89^{th} percentile of severity) and sociability skills (60-69^{th} percentile of severity).

Laboratory analyses did not reveal elevated levels of mercury or elevated levels of at least one androgen. Specifically, undetectable levels of mercury were present in Child D's urine and minimal levels of mercury were in Child D's blood (1.5 μg/L, reference range=0.0-14.9 μg/L). Additionally, analyses of Child D's blood androgen metabolites revealed a serum testosterone=153 ng/dL (age- and sex-adjusted LabCorp reference range=0-350 ng/dL) and serum/plasma DHEA=291 ng/dL (age- and sex-adjusted LabCorp reference range=183-383 ng/dL) within their respective reference ranges.

After extensive discussions with his parents concerning the risks, benefits, and alternative treatments available, a decision was made to place Child D on a course of LUPRON® therapy.

The therapy was begun an IM injection of Pediatric LUPRON DEPOT® 15 mg. This was augmented with 0.4 ml of daily LUPRON® injected subcutaneously. This daily dose was gradually increased in 0.1 ml increments as indicated by clinical and laboratory monitoring to a dose of 0.7 ml per day (since Child D weighed approximately 273 pounds (124 kg) this is 32 micrograms per kilogram).

With the advent of his LUPRON® therapy, Child D's serum testosterone declined, namely, to 35 ng/dL (from 153 ng/dL) after several months of receiving the treatment.

Child D was re-evaluated after 104 days on LUPRON® therapy using the ATEC which then showed significant improvements as follows: overall impairments (40-49^{th} percentile of severity, down from 80-89^{th} percentile of severity), impairments in his speech/language/communications skills (60-69^{th} percentile of severity, down from 70-79^{th} percentile of severity), sensory cognitive awareness skills (40-49^{th} percentile of severity down from 50-59^{th} percentile of severity), health/physical/behavior skills (60-69^{th} percentile of severity down from 80-89^{th} percentile of severity) and sociability skills (20-29^{th} percentile of severity down from 60-69^{th} percentile of severity). His parents and his educators reported major improvements in attention, cognitive awareness, and receptive language skills.

### Example 7

### Child E

The patient, Child E, was an eleven year old white female who was seen by a physician for work up and possible treatment her autism spectrum disorder and Attention-Deficit-Hyperactivity-Disorder (“ADHD”). Child E was the product of a full term uneventful pregnancy. The patient's parents were both in good health at the time of Child's E delivery. The patient was the product of a full term spontaneous vaginal delivery. There were no significant birthing complications and patient's neonatal course was completely uneventful. The patient weighed six pounds and twelve ounces at birth and had APGAR scores of eight and ten respectively at one and five minutes after birth. Child E met all of her developmental milestones, both physical and mental, for her first five years of life but was observed to have to have significant difficulties with attention, concentration and hyperactivity and was diagnosed as having ADHD at five years of age. The patient was treated with Adderall XR® (Shire US Inc., Wayne, Pa. Adderall XR® is an amphetamine product combining the neutral sulfate salts of dextroamphetamine and amphetamine, with the dextro isomer of amphetamine saccharate and d, 1-amphetamine aspartate monohydrate), which seemed to help her symptoms to some extent. She has been mainstreamed and is currently at grade level in public school but continues to have considerable difficulty with socialization, attention and hyperactivity. She also showed some mild signs of precocious puberty and had fully developed pubic hair by the age of eight. She began to have periods around her tenth birthday. Since then her periods have been very irregular and painful. The patient completed the full schedule of recommended childhood vaccinations almost all of which had full dose Thimerosal (mercury).

Genetic testing for chromosomal anomalies, Fragile X, chromosomal abnormalities, plasma amino acid abnormalities, and subtelomere chromosomal anomalies were all normal. Abdominal, thyroid and pelvic sonograms were all within normal limits. MTHFR DNA analysis showed her to carry two SNP mutations (C677T and A1298C) making her highly susceptible to mercury toxicity. Her serum testosterone was within normal limits at 26, with her age and sex specific reference range of 0-30, however her free testosterone was high at 0.61 with her age and sex specific reference range of 0.10 to 0.52 and her percent free testosterone was also high at 2.33 with her age and sex specific cutoff at 1.00 to 1.90. Her screen for PCBs and pesticide exposure were all negative. Her thyroid panel was within normal limits. Her urophorphyrin and hexacarboxylphorphyrins were both elevated. The rest of her laboratory screens were within normal limits.

The patient was treated with LUPRON® Pediatric Depot 15 mg intramuscular injections every 28 days, 3.5 mg LUPRON® by subcutaneous injection daily and low dose birth control pills.

The patient responded well to this regiment with major improvement in socialization, attention and reduced hyperactivity as reported by the patient and her parents. Her periods also became regular and without pain.

One skilled in the art would readily appreciate that the present invention is well adapted to carry out the objects and obtain the ends and advantages mentioned, as well as those inherent therein. The molecular complexes and the methods, procedures, treatments, molecules, specific compounds described herein are presently representative of preferred embodiments, are exemplary, and are not intended as limitations on the scope of the invention. It will be readily apparent to one skilled in the art that varying substitutions and modifications may be made to the invention disclosed herein without departing from the scope and spirit of the invention.

All patents and publications mentioned in the specification are indicative of the levels of those skilled in the art to which the invention pertains. All patents and publications are herein incorporated by reference to the same extent as if each individual publication was specifically and individually indicated to be incorporated by reference.

The invention illustratively described herein suitably may be practiced in the absence of any element or elements, limitation or limitations which is not specifically disclosed herein. Thus, for example, in each instance herein any of the terms “comprising,” “consisting essentially of” and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

In addition, where features or aspects of the invention are described in terms of Markush groups, those skilled in the art will recognize that the invention is also thereby described in terms of any individual member or subgroup of members of the Markush group. For example, if X is described as selected from the group consisting of bromine, chlorine, and iodine, claims for X being bromine and claims for X being bromine and chlorine are fully described.

We claim:

1. A method of treating a subject suffering from autism, the method comprising the step of: a) administering to the subject a pharmaceutically effective amount of at least one luteinizing hormone releasing hormone composition to treat the autism, wherein the at least one luteinizing hormone releasing hormone composition is administered in a sufficient amount and over a sufficient period of time to control clinical symptoms of autism to a desired level, and wherein when the subject is younger than 18 years and said luteinizing hormone releasing hormone composition comprises leuprolide acetate, and the subject is administered a dosage of the composition of at least about 20 ug/kg per day for at least 28 days or said leuprolide acetate dosage is administered via a slow release formulation that releases said leuprolide acetate daily dosage over a 28 day period, and
wherein when the subject is 18 years old or older than 18 years said luteinizing hormone releasing hormone composition comprises leuprolide acetate, and the subject is administered a dosage of leuprolide acetate of at least about 0.3 mg per day for at least 28 days or said leuprolide acetate dosage is administered via a slow release formulation that releases said leuprolide acetate daily dosage over a 28 day period.

2. The method of claim 1, wherein said method further comprises the step of: b) determining control of clinical symptoms of from an ATEC (Autism, Treatment, Evaluation, Checklist) Form, wherein the development of the form is credited to the Autism Research Institute of San Diego, Calif., Bernard Rimland and Stephen M. Edelson, wherein step b) can be performed before and after step a).

3. The method of claim 1, wherein the subject has an elevated level of at least one androgen when compared to a reference level for the at least one androgen in a subject having approximately the same age.

4. The method of claim 3, wherein step a) is repeated as necessary to reduce the level of the at least one androgen in the subject and treat the autism in the subject.

5. The method of claim 1, further comprising the step of administering to the subject a pharmaceutically effective amount of at least one antiandrogenic hormone prior to step a), step b) or step a) and step b).

6. The method of claim 5, wherein the at least one antiandrogenic hormone is selected from the group consisting of cyproterone acetate, finasteride, bicalutamide, novaldex, nilandron, flutamide, progesterone, spironolactone, and fluconazole.

7. The method of claim 1, further comprising the step of administering to the subject a pharmaceutically effective amount of at least one androgen compound prior to step a), step b) and step a) and step b).

8. The method of claim 1, further comprising the step of administering to the subject, if the subject is a pubertal age female, a pharmaceutically effective amount of at least one estrogen compound prior to step a), step b) or step a) and step b).

9. The method of claim 8, wherein said step of administering a pharmaceutically effective amount of at least one estrogen compound is repeated as necessary to treat the autism.

10. The method of claim 8, further comprising administering to the subject a pharmaceutically effective amount of at least one progesterone compound or at least one progestin compound with the at least one estrogen compound.

11. The method of claim 8, wherein said step of administering a pharmaceutically effective amount of at least one progesterone compound or at least one progestin compound with the at least one estrogen compound is repeated as necessary to treat the autism.

12. The method of claim 1, wherein the subject is under the age of 18 years and is a male or female.

13. The method of claim 12, wherein the subject is a male child.

14. The method of claim 13, wherein the male child has been diagnosed with precocious puberty.

15. The method of claim 1, wherein when the subject is a male child that also been diagnosed with precocious puberty said administration is sufficient to control symptoms of autism in addition to symptoms associated with precocious puberty.

16. The method of claim 1, wherein when the subject is a male child that has been diagnosed with precocious puberty as well and exhibits masturbation associated therewith, said administration is sufficient to control symptoms of autism in addition to symptoms associated with precocious puberty.

17. The method of claim 12, wherein the subject is a male child.

18. The method of claim 12, wherein the male child has been diagnosed with precocious puberty.

19. The method of claim 1, wherein the at least one luteinizing hormone releasing hormone composition comprises a peptide having the formula
(Pyr)Glu-R¹-Trp-Ser-R²—R³—R⁴-Arg-Pro-R⁵
wherein R¹ comprises His, Tyr, Trp, or p-NH₂-Phe, R² is Tyr or Phe, R³ comprises Gly or D type amino acid residue, R⁴ comprises Leu, lie, or Nle, and R⁵ comprises Gly-NH—R⁶, NH—R⁷, or —NH—CO—NH₂, wherein R⁶ comprises H or an alkyl group, and R⁷ comprises —H, an amino group, an alkyl group, or a ureido group, wherein when the at least one luteinizing hormone releasing hormone composition administered comprises at least one peptide other than leuprolide acetate, administration of a sufficient amount of the composition over a sufficient period of time will give the same results as said administration of said leuprolide acetate alone.

20. The method of claim 2, wherein the subject has an elevated level of at least one androgen when compared to a reference level for the at least one androgen in a subject having approximately the same age.

21. The method of claim 20, wherein step a) is repeated as necessary to reduce the level of the at least one androgen in the subject and treat the autism in the subject.

22. The method of claim 2, further comprising the step of administering to the subject a pharmaceutically effective amount of at least one antiandrogenic hormone prior to step a), step b) or step a) and step b).

23. The method of claim 22, wherein the at least one antiandrogenic hormone is selected from the group consisting of cyproterone acetate, finasteride, bicalutamide, novaldex, nilandron, flutamide, progesterone, spironolactone, and fluconazole.

24. The method of claim 2, further comprising the step of administering to the subject a pharmaceutically effective amount of at least one androgen compound prior to step a), step b) and step a) and step b).

25. The method of claim 2, further comprising the step of administering to the subject, if the subject is a pubertal age female, a pharmaceutically effective amount of at least one estrogen compound prior to step a), step b) or step a) and step b).

26. The method of claim 25, wherein said step of administering a pharmaceutically effective amount of at least one estrogen compound is repeated as necessary to treat the autism.

27. The method of claim 2, wherein the subject is under the age of 18 years and is a male.

28. The method of claim 27, wherein the male child has been diagnosed with precocious puberty, wherein said administration is sufficient to control symptoms of autism in addition to symptoms associated with precocious puberty.

29. The method of claim 2, wherein when the subject is a male child that has been diagnosed with precocious puberty as well and exhibits masturbation associated therewith, said administration is sufficient to control symptoms of autism in addition to symptoms associated with precocious puberty.

30. A method of treating a subject suffering from autism, the method comprising the step of administering to the subject younger than the age of 18 a pharmaceutically effective amount of at least one luteinizing hormone releasing hormone composition to treat the autism,
wherein the at least one luteinizing hormone releasing hormone composition is administered in a sufficient amount and over a sufficient period of time to control clinical symptoms of autism to a desired level,
wherein when said luteinizing hormone releasing hormone composition comprises leuprolide acetate, the subject is administered a dosage of the composition of at least about 20 ug/kg per day for at least 28 days or said leuprolide acetate dosage is administered via a slow release formulation that releases said leuprolide acetate daily dosage over a 28 day period, and
wherein the at least one luteinizing hormone releasing hormone composition comprises a peptide having the formula
(Pyr)Glu-R¹-Trp-Ser-R²—R³—R⁴-Arg-Pro-R⁵
wherein R¹ comprises His, Tyr, Trp, or p-NH₂-Phe, R² is Tyr or Phe, R³ comprises Gly or D type amino acid residue, R⁴ comprises Leu, lie, or Nle, and R⁵ comprises Gly-NH—R⁶, NH—R⁷, or —NH—CO—NH₂, wherein R⁶ comprises H or an alkyl group, and R⁷ comprises —H, an amino group, an alkyl group, or a ureido group, wherein when the at least one luteinizing hormone releasing hormone composition administered comprises at least one peptide other than leuprolide acetate, administration of a sufficient amount of the composition over a sufficient period of time will give the same results as said administration of said leuprolide acetate alone.
